(19) United States
(12) Patent Application Publication
(10) Pub. No.: US 2026/0234634 A1
(43) Pub. Date: Aug. 13, 2026

(54) UNG/UDG INHIBITION IN BRCA-ASSOCIATED CANCER

(71) Applicants: INSTITUT CURIE, PARIS (FR); INSTITUT NATIONAL DE LA SANTE ET DE LA RECHERCHE MEDICALE, PARIS (FR)

(72) Inventors: Raphaël CECCALDI, PARIS (FR); Yucel HATICE, PARIS (FR); Daniele MUSIANI, PARIS (FR)

(21) Appl. No.: 19/156,195

(22) PCT Filed: Mar. 7, 2024
(86) PCT No.: PCT/EP2024/056056

§ 371 Date: Aug. 13, 2025

(30) Foreign Application Priority Data
Mar. 7, 2023 (EP) 23305303.2

(51) Int. Cl. C12N 15/113; A61K 31/55; A61P 35/00

(52) U.S. Cl. CPC C12N 15/1137; A61K 31/55; A61P 35/00; C12Y 302/02027; C12N 2310/14; C12N 2320/31

(57) ABSTRACT

Methods and pharmaceutical compositions for the treatment of resistant HRD cancer, particularly resistant BRCA-associated cancer, chemo-resistant HRD cancer and chemo-resistant BRCA-associated cancer.

### FIELD OF THE INVENTION

The invention relates to methods and pharmaceutical compositions for the treatment of resistant HRD cancer, particularly resistant BRCA-associated cancer, chemo-resistant HRD cancer and chemo-resistant BRCA-associated cancer.

### BACKGROUND OF THE INVENTION

In the last decades, many efforts have been undertaken to gain mechanistic insights into the synthetic lethality between PARP1 and BRCA1/2 genes (Maya-Mendoza et al., Nature 559, pages 279-284, 2018; Hanzlikova et al., Nature 559, pages 279-284, 2018). Deficiencies in homologous recombination (HR)-mediated DNA repair occur mainly through genetic inactivation of BRCA1 and BRCA2 (BRCA1/2) genes and play a role in the initiation and progression of many tumor types. Defective HR (HRD) causes genomic instability and hyper-dependence on alternative DNA repair mechanisms for survival, setting the stage for synthetic lethality-based targeted therapy, as exemplified by the extreme sensitivity of HRD tumors to poly (ADP-ribose) polymerase inhibitors (PARPi) (Bryant et al., Nature 434, pages 913-917, 2005; Farmer et al., Nature 434, pages 917-921, 2005). This lethal interaction is successfully exploited in the clinic since the approval of PARP inhibitors (PARPi) for the treatment of BRCA1/2-mutated tumors (Patel et al., Oncogene 40, pages 3001-3014, 2021), also referred as homologous recombination (HR)-deficient tumors (HRD). Various trials have been devised to evaluate PARPi effectiveness for patients with breast, ovarian, and prostate tumors harboring BRCA1/2 mutations (Pujade-Lauraine et al., The Lancet. Oncology 18, Issue 9, pages 1274-1284, 2017). However, PARPi and other chemotherapeutics have shown limited effectiveness in achieving HRD cancer remission, notably because drug resistance emerges and resistance to chemotherapy is emerging as the major obstacle to clinic effectiveness (Gogola et al., Annual Review of Cancer Biology 3, pages 235-254, 2019). Hence, no therapeutic options are left for these patients, stressing the need for alternative therapeutic options (Konstantinopoulos et al., Cancer Discov 5, pages 1137-1154, 2015).

The inventors have previously identified that both NMNAT1 (nicotinamide mononucleotide adenylyltransferase 1) and the NAD+-dependent deacetylase SIRT6 are synthetically lethal with BRCA1/2 through their function in base-excision repair. The enzyme NMNAT1, key to nuclear NAD biogenesis, maintains genomic stability in BRCA1/2-mutated cells. Targeting the SIRT6 axis sensitizes cells to PARPi, indicating that this pathway is not epistatic with PARP1.

Consequently, inhibition of NMNAT1 or SIRT6 not only kills HRD cells, but also the PARPi-resistant and platinium-resistant ones, regardless the mechanism of resistance, demonstrating a surprising effect on killing resistant HRD cells, including those with acquired drug resistance. To find alternative curative options for HRD cancer including BRCA1/2-mutated tumors, and the ones that have acquired PARPi resistance, the inventors investigated further the action of the NMNAT1/SIRT6 pathway.

Uracil DNA glycosylase (UNG) is a protein member of the uracil-DNA glycosylases (UDGs) family. Four UDGs (namely UNG, SMUG1, TDG and MBD4) have been characterized in human cells with different cellular localizations and catalytic efficiencies to ensure efficient uracil recognition and excision. One important function of uracil-DNA glycosylases is to prevent mutagenesis by eliminating uracil from DNA molecules by cleaving the N-glycosylic bond and initiating the base-excision repair (BER) pathway. UNG is an important player in DNA damage localization and repair of uracil accumulation in telomeres (Baquero et al, Mol Oncol 13, Issue 5, pages 1110-1120, 2019). UNG initiates BER activity, and its depletion has been reported to restore sensitivity to some DNA damaging agents such as stalled fork inducing agents in BRCA2-deficient cells (Pathania, Grantome NIH, project 1R15CA235436-01A1). In addition, UNG knockdown can induce apoptosis in prostate cancer cell lines, reduce cell proliferation and increase cellular sensitivity to genotoxic stress. Others have observed that colon cancer cells lacking UNG can be hypersensitive to pemetrexed-induced uracil accumulation, resulting in cell cycle arrest, DNA double-strand break formation, and apoptosis (Pulukuri et al., Mol Cancer Res 7, pages 1285-1293, 2009; Weeks et al., Mol Cancer Ther 12, pages 2248-2260, 2013).

The inventors have demonstrated that the role of UNG in the maintenance of genome stability is crucial for the survival of BRCA1/2-deficient cells and that UNG is also synthetically lethal with BRCA1/2 through its function in base-excision repair. Depletion of UNG reduces survival of HRD cells and PARPi-resistant cells, and sensitizes HR proficient cells to PARPi. UNG is the target of NMNAT1/SIRT6 pathway and functions downstream of SIRT6 in the survival of BRCA1/2-mutated tumors. As inhibition of NMNAT1 or SIRT6 kills HRD cells and PARPi- and platinium resistant cells, regardless the mechanism of resistance, inhibition of UNG should be able to target also PARPi- and platinium-resistant HRD cells.

### SUMMARY OF THE INVENTION

The invention relates to methods and pharmaceutical compositions for the treatment of resistant HRD cancer, particularly resistant BRCA-associated cancer, chemo-resistant HRD cancer and chemo-resistant BRCA-associated cancer. In particular, the invention is defined by the claims.

### DETAILED DESCRIPTION OF THE INVENTION

The inventors investigated the role of UNG in HRD cancer, particularly in chemo-resistant BRCA-associated cancer, and in NMNAT1/SIRT6 pathway.

Previously, the inventors demonstrated that SIRT6 inhibition kills BRCA1 and BRCA2-mutated tumor cells but does not affect the survival of non-BRCA mutated cells. The inventors also demonstrated that deletion of SIRT6 sensitized cells to PARPi. In particular, inhibition of SIRT6 kills PARP-inhibitor and cisplatin-resistant BRCA1 and BRCA2-mutated tumors, including those with somatic reversion of the BRCA1/2 mutations. Targeting SIRT6 thus kills chemo-resistant HRD cells, particularly PARPi-resistant HRD cells.

The inventors have now demonstrated that the Uracil DNA glycosylase (UNG) is also synthetically lethal with BRCA1/2 through its function in base-excision repair and that UNG is the target of NMNAT1/SIRT6 pathway. UNG functions downstream of SIRT6 in the survival of BRCA1/2-mutated tumors.

The inventors have further demonstrated that targeting UNG-mediated uracil excision leads to SMUG1 chromatin trapping and HMCES (5-hydroxymethylcytosine binding, ES cell specific) crosslinking to the SMUG1-generated abasic (AP) sites, which hinders their processing by APE1. This blocks replication fork (RF) progression and creates DNA lesions that trigger HR-mediated repair. Blocking or overcoming UNG excision capability leads to dramatic consequences in HRD cells, where SMUG1 trapping results in under-replicated DNA that—when propagated through mitosis—leads to chromosomal fragmentation, rearrangements and ultimately cell death. These results unveil uracil excision by UNG as a novel vulnerability of HRD cancer tumors with some potential to tackle PARPi resistance.

Altogether, the present invention highlights the role of UNG inhibitors in HRD cancer and the use of UNG inhibitors in the treatment of resistant HRD cancer, particularly resistant BRCA-associated cancer, chemo-resistant HRD cancer and chemo-resistant BRCA-associated cancer including BRCA-associated cancer with acquired drug resistance to mono- or combination therapy with PARPi.

Accordingly, the invention relates to the targeting of UNG in the treatment of resistant HRD cancer, particularly resistant BRCA-associated cancer, chemo-resistant HRD cancer and chemo-resistant BRCA-associated cancer.

### UNG Inhibitors

Accordingly, in a first aspect, the invention relates to a Uracil DNA glycosylase (UNG) inhibitor for use in the treatment of resistant Homologous Recombination Deficiency (HRD) cancer, wherein the UNG inhibitor is a molecule capable of silencing the gene expressing the UNG enzyme or a molecule inhibiting UNG enzymatic properties.

In some embodiment, the invention relates to UNG inhibitor wherein the resistant HRD cancer is resistant BRCA-associated cancer, chemo-resistant HRD cancer, chemo-resistant BRCA-associated cancer or metastatic resistant HRD cancer.

In some embodiment, the invention relates to UNG inhibitor wherein the resistant HRD cancer is PARPi resistant BRCA-associated cancer or cisplatin resistant BRCA-associated cancer, including those with somatic reversion of the BRCA mutation and HR restoration.

As used herein, the terms “subject”, “individual” or “patient” are interchangeable and refer to a mammal. Typically, a subject according to the invention refers to any subject, preferably human. In a particular embodiment, the term “subject” refers to a subject afflicted or at risk to be afflicted with cancer. In a particular embodiment, the term “subject” refers to a subject afflicted or at risk to be afflicted with HRD cancer, particularly BRCA-associated cancer. In a particular embodiment, the term “subject” refers to a subject afflicted or at risk to be afflicted with resistant HRD cancer. In some embodiment, the term “subject” refers to a subject afflicted or at risk to be afflicted with resistant BRCA-associated cancer. In some embodiment, the term “subject” refers to a subject afflicted or at risk to be afflicted with chemo-resistant HRD cancer. In some embodiment, the term “subject” refers to a subject afflicted or at risk to be afflicted with chemo-resistant BRCA-associated cancer. In some embodiment, the term “subject” refers to a subject afflicted or at risk to be afflicted with metastatic resistant HRD cancer.

In some embodiment, the term “subject” refers to a subject afflicted or at risk to be afflicted with chemo-resistant BRCA-associated cancer such as chemo-resistant HRD cancer and/or BRCA-deficiency cancer (basal-like, luminal, and HER2-overexpressing breast carcinomas and other cancers) and breast, ovarian, prostate, pancreatic or any other type of tumors harboring BRCA1/2 mutations or BRCA expression deficiency. In a particular embodiment, the term “subject” refers to a subject afflicted or at risk to be afflicted with PARPi resistant BRCA-associated cancer or cisplatin resistant BRCA-associated cancer.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “cancer” refers to any cancer that may affect any one of the following tissues or organs: breast; liver; kidney; heart, mediastinum, pleura; floor of mouth; lip; salivary glands; tongue; gums; oral cavity; palate; tonsil; larynx; trachea; bronchus, lung; pharynx, hypopharynx, oropharynx, nasopharynx; esophagus; digestive organs such as stomach, intrahepatic bile ducts, biliary tract, pancreas, small intestine, colon; rectum; urinary organs such as bladder, gallbladder, ureter; rectosigmoid junction; anus, anal canal; skin; bone; joints, articular cartilage of limbs; eye and adnexa; brain; peripheral nerves, autonomic nervous system; spinal cord, cranial nerves, meninges; and various parts of the central nervous system; connective, subcutaneous and other soft tissues; retroperitoneum, peritoneum; adrenal gland; thyroid gland; endocrine glands and related structures; female genital organs such as ovary, uterus, cervix uteri; corpus uteri, vagina, vulva; male genital organs such as penis, testis and prostate gland; hematopoietic and reticuloendothelial systems; blood; lymph nodes; thymus.

The term “cancer” according to the invention comprises leukemias, seminomas, melanomas, teratomas, lymphomas, non-Hodgkin lymphoma, neuroblastomas, gliomas, adenocarninoma, mesothelioma (including pleural mesothelioma, peritoneal mesothelioma, pericardial mesothelioma and end stage mesothelioma), rectal cancer, endometrial cancer, thyroid cancer (including papillary thyroid carcinoma, follicular thyroid carcinoma, medullary thyroid carcinoma, undifferentiated thyroid cancer, multiple endocrine neoplasia type 2A, multiple endocrine neoplasia type 2B, familial medullary thyroid cancer, pheochromocytoma and paraganglioma), skin cancer (including malignant melanoma, basal cell carcinoma, squamous cell carcinoma, Karposi's sarcoma, keratoacanthoma, moles, dysplastic nevi, lipoma, angioma and dermatofibroma), nervous system cancer, brain cancer (including astrocytoma, medulloblastoma, glioma, lower grade glioma, ependymoma, germinoma (pinealoma), glioblastoma multiform, oligodendroglioma, schwannoma, retinoblastoma, congenital tumors, spinal cord neurofibroma, glioma or sarcoma), skull cancer (including osteoma, hemangioma, granuloma, xanthoma or osteitis deformans), meninges cancer (including meningioma, meningiosarcoma or gliomatosis), head and neck cancer (including head and neck squamous cell carcinoma and oral cancer (such as, e.g., buccal cavity cancer, lip cancer, tongue cancer, mouth cancer or pharynx cancer)), lymph node cancer, gastrointestinal cancer, liver cancer (including hepatoma, hepatocellular carcinoma, cholangiocarcinoma, hepatoblastoma, angiosarcoma, hepatocellular adenoma and hemangioma), colon cancer, stomach or gastric cancer, esophageal cancer (including squamous cell carcinoma, larynx, adenocarcinoma, leiomyosarcoma or lymphoma), colorectal cancer, intestinal cancer, small bowel or small intestines cancer (such as, e.g., adenocarcinoma lymphoma, carcinoid tumors, Karposi's sarcoma, leiomyoma, hemangioma, lipoma, neurofibroma or fibroma), large bowel or large intestines cancer (such as, e.g., adenocarcinoma, tubular adenoma, villous adenoma, hamartoma or leiomyoma), pancreatic cancer (including ductal adenocarcinoma, insulinoma, glucagonoma, gastrinoma, carcinoid tumors or vipoma), ear, nose and throat (ENT) cancer, breast cancer (including HER2-enriched breast cancer, luminal A breast cancer, luminal B breast cancer and triple negative breast cancer), cancer of the uterus (including endometrial cancer such as endometrial carcinomas, endometrial stromal sarcomas and malignant mixed Müllerian tumors, uterine sarcomas, leiomyosarcomas and gestational trophoblastic disease), ovarian cancer (including dysgerminoma, granulosa-theca cell tumors and Sertoli-Leydig cell tumors), cervical cancer, vaginal cancer (including squamous-cell vaginal carcinoma, vaginal adenocarcinoma, clear cell vaginal adenocarcinoma, vaginal germ cell tumors, vaginal sarcoma botryoides and vaginal melanoma), vulvar cancer (including squamous cell vulvar carcinoma, verrucous vulvar carcinoma, vulvar melanoma, basal cell vulvar carcinoma, Bartholin gland carcinoma, vulvar adenocarcinoma and erythroplasia of Queyrat), genitourinary tract cancer, kidney cancer (including clear renal cell carcinoma, chromophobe renal cell carcinoma, papillary renal cell carcinoma, adenocarcinoma, Wilm's tumor, nephroblastoma, lymphoma or leukemia), adrenal cancer, bladder cancer, urethra cancer (such as, e.g., squamous cell carcinoma, transitional cell carcinoma or adenocarcinoma), prostate cancer (such as, e.g., adenocarcinoma or sarcoma) and testis cancer (such as, e.g., seminoma, teratoma, embryonal carcinoma, teratocarcinoma, choriocarcinoma, sarcoma, interstitial cell carcinoma, fibroma, fibroadenoma, adenomatoid tumors or lipoma), lung cancer (including small cell lung carcinoma (SCLC), non-small cell lung carcinoma (NSCLC) including squamous cell lung carcinoma, lung adenocarcinoma (LUAD), and large cell lung carcinoma, bronchogenic carcinoma, alveolar carcinoma, bronchiolar carcinoma, bronchial adenoma, lung sarcoma, chondromatous hamartoma and pleural mesothelioma), sarcomas (including Askin's tumor, sarcoma botryoides, chondrosarcoma, Ewing's sarcoma, malignant hemangioendothelioma, malignant schwannoma, osteosarcoma and soft tissue sarcomas), soft tissue sarcomas (including alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, plexiform fibrohistiocytic tumor, rhabdomyosarcoma, synovial sarcoma and undifferentiated pleomorphic sarcoma, cardiac cancer (including sarcoma such as, e.g., angiosarcoma, fibrosarcoma, rhabdomyosarcoma or liposarcoma, myxoma, rhabdomyoma, fibroma, lipoma and teratoma), bone cancer (including osteogenic sarcoma, osteosarcoma, fibrosarcoma, malignant fibrous histiocytoma, chondrosarcoma, Ewing's sarcoma, malignant lymphoma and reticulum cell sarcoma, multiple myeloma, malignant giant cell tumor chordoma, osteochronfroma, osteocartilaginous exostoses, benign chondroma, chondroblastoma, chondromyxoid fibroma, osteoid osteoma and giant cell tumors), hematologic and lymphoid cancer, blood cancer (including acute myeloid leukemia, chronic myeloid leukemia, acute lymphoblastic leukemia, chronic lymphocytic leukemia, myeloproliferative diseases, multiple myeloma and myelodysplasia syndrome), Hodgkin's disease, non-Hodgkin's lymphoma and hairy cell and lymphoid disorders, and the metastases thereof.

The term “homologous recombination deficiency cancer” or “HRD cancer” has its general meaning in the art and refers to cancer displaying defective homologous recombination (HRD)-mediated DNA repair which causes genomic instability and hyper-dependence on alternative DNA repair mechanisms for survival. Thus, the term “HRD cancer” refers to a cancer displaying defective homologous recombination due to alteration of genes including PALB2, CHEK2, ATM, BARD1, RAD51C, RAD51D, FANCC, BRIP1, FANCM, XRCC2, or any other genes involved in HR DNA repair pathway. The HR status of a cancer can be determined by any well-known methods in the art, for example by probing the genome for HRD-related genomic signatures or by using a commercial HRD diagnostic test such as Myriad myChoice®. The term “HRD cancer” includes but is not limited to BRCA-associated cancer. Conversely, a BRCA-associated cancer is not necessarily a HRD cancer.

The term “BRCA-associated cancer” has its general meaning in the art and refers to cancer associated with BRCA mutation or BRCA expression deficiency. The term “BRCA-associated cancer” refers to cancer selected from cancer associated with BRCA1 and/or BRCA2 mutation, cancer associated with BRCA1 and/or BRCA2 expression deficiency, and homologous recombination deficiency (HRD) cancer with BRCA-deficiency (such as basal-like, luminal, and HER2-overexpressing carcinomas, breast, ovarian, and prostate tumors harboring BRCA1/2 mutations and other cancers). As used herein “BRCA1/2” denotes BRCA1 and/or BRCA2, more particularly BRCA1 and BRCA2. In some embodiment, the term “BRCA-associated cancer” refers to breast cancer, ovary cancer, cervix cancer, pancreas cancer, lung cancer, head and neck cancer and melanoma with BRCA1 and/or BRCA2 mutation or BRCA1 and/or BRCA2 expression deficiency. In some embodiment, the term “BRCA-associated cancer” refers to metastatic BRCA-associated cancer.

The term “resistant HRD cancer” has its general meaning in the art and refers to HRD cancer resistant to treatment such as HRD cancer resistant to chemotherapy, radiotherapy and other cancer therapy. The term “resistant HRD cancer” also refers to resistant BRCA-associated cancer, chemo-resistant HRD cancer, chemo-resistant BRCA-associated cancer such as PARP inhibitor (PARPi) resistant BRCA-associated cancer, PARPi-resistant HRD tumors including tumors with somatic reversion of BRCA1/2 mutation and subsequent HR restoration, cisplatin resistant BRCA-associated cancer and cisplatin-resistant BRCA1 and BRCA2-mutated tumors including tumors with somatic reversion of BRCA1/2 mutation and subsequent HR restoration. In some embodiment, the term “resistant HRD cancer” refers to metastatic resistant HRD cancer.

The term “PARP inhibitor” or “PARPi” has its general meaning in the art and refers to PARP inhibitor such as olaparib, rucaparib, niraparib and talazoparib. The term “PARP inhibitor” also refers to PARP inhibitor such iniparib, veliparib, Pamiparib (BGB-290), CEP 9722, E7016 and 3-Aminobenzamide.

The term “SIRT6” refers to NAD-Dependent Protein Deacetylase Sirtuin-6. Previously, the inventors demonstrated that SIRT6 inhibition kills BRCA1 and BRCA2-mutated tumor cells but does not affect the survival of non-BRCA mutated cells. The inventors also demonstrated that deletion of SIRT6 sensitized cells to PARPi. The inventors also demonstrated that inhibition of SIRT6 kills PARP-inhibitor and cisplatin-resistant BRCA1 and BRCA2-mutated tumors, including those with somatic reversion of the BRCA1/2 mutations, and show that targeting SIRT6 kills chemo-resistant HRD cells, particularly PARPi-resistant HRD cells.

The term “NMNAT1” has its general meaning in the art and refers to a Nicotinamide Nucleotide Adenylyltransferase 1, also known as Nicotinamide/nicotinic acid mononucleotide adenylyltransferase 1 (Protein Accession number Q9HAN9).

The term “UNG” or “UDG” refers to Uracil DNA glycosylase enzymes and coding gene. This gene encodes one of several uracil-DNA glycosylases. One important function of uracil-DNA glycosylases is to prevent mutagenesis by eliminating uracil from DNA molecules by cleaving the N-glycosylic bond and initiating the base-excision repair (BER) pathway. Alternative promoter usage and splicing of this gene leads to two different isoforms: the mitochondrial UNG1 and the nuclear UNG2. As used herein, by “UNG”, it is meant any one of the two UNGs, or both (i.e. UNG1 and/or UNG2).

In some embodiment, the UNG inhibitor is a small organic molecule, a polypeptide, an aptamer, an oligonucleotide or an antibody.

As used herein, the term “UNG inhibitor” refers to any compound selected from the group consisting of but not limited to compounds targeting Uracil DNA glycosylase. The term “UNG inhibitor” refers to compounds that bind to UNG and function as potent antagonists of UNG. The term “UNG inhibitor” has its general meaning in the art and refers to a compound that selectively inactivates UNG. Typically, a UNG inhibitor is a small organic molecule, a polypeptide, an aptamer, an oligonucleotide (antisense oligonucleotides, siRNA, shRNA, DNA and RNA aptamers), or an antibody. Some UNG inhibitors are known in the art as such as described in WO 2021/087246 A1, WO 2006/135763, WO 2006/135763, U.S. Pat. No. 6,177,437 and WO 1998/039334.

The term “UNG inhibitor” refers to any compound selected from but not limited to the non-protein uracil-DNA glycosylase inhibitor (npUGI) as follow:

In some embodiments, the small molecule inhibitor of UDG is a compound of formula (I):

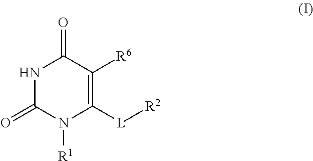

or a pharmaceutically acceptable salt thereof,
wherein:
R1 is H, a furanose carbohydrate, a pyranose carbohydrate, a carbohydrate mimetic, C1-16alkyl, C1-16alkenyl, C1-16alkynyl, C1-16alkoxy, or C6-20aryl, wherein the furanose carbohydrate, or a derivative thereof, pyranose carbohydrate, or a derivative thereof, carbohydrate mimetic, C1-16alkyl, C1-16alkenyl, C1-16alkynyl, C1-16alkoxy, or C6-20aryl is independently optionally substituted with one or more substituents selected from the group of hydroxyl, halo, cyano, NO2, N(R4)(R5), C1-16alkoxy, or C6-20aryl,
wherein the C6-20aryl is further optionally substituted with one or more substituents selected from the group of C1-16alkyl, C1-16alkenyl, C1-16alkynyl, C1-16alkoxy, hydroxyl, halo, cyano, NO2, or N(R4)(R5);
L is O, S, or *—N(R3)-**, wherein R3 is H or C1-16alkyl, and ** indicates the point of attachment to the R2 moiety and * indicates the point of attachment to the remainder of the molecule;
R2 is H, N(R4)(R5), or C6-20aryl, wherein the C6-20aryl is independently substituted with one or more substituents selected from the group of C1-16alkyl, C1-16alkenyl, C1-16alkynyl, or C1-16alkoxy;
R4 and R5 are each independently H or C6-20aryl; and
R6 is H or halo.

In some embodiments, the small molecule inhibitor of UDG is a compound of formula (II):

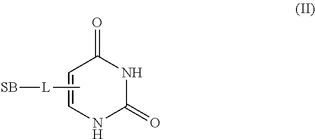

wherein
L is a linker, e.g., a linker comprising an imine or oxime moiety;
SB is a binding element, e.g., a binding element comprising a phenyl group.

In some embodiments, the small molecule inhibitor of UDG is a compound of formula (IIa):

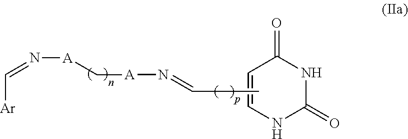

wherein
A are independently —O—, —CH2- ou single bond
Ar is an aromatic or heteroaromatic group

In some embodiments, the small molecule inhibitor of UDG is a compound from the followings:

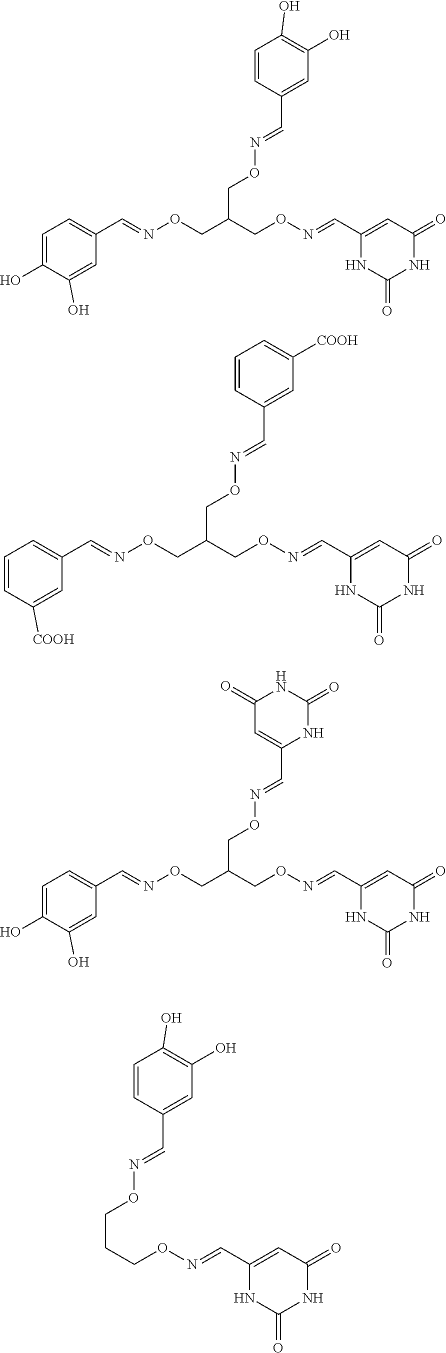 [SMILES: O=c1cc(/C=N/OCC(CO/N=C/c2ccc(O)c(O)c2)CO/N=C/c2ccc(O)c(O)c2)[nH]c(=O)[nH]1 ; O=C(O)c1cccc(/C=N/OCC(CO/N=C/c2cccc(C(=O)O)c2)CO/N=C/c2cc(=O)[nH]c(=O)[nH]2)c1 ; O=c1cc(/C=N/OCC(CO/N=C/c2ccc(O)c(O)c2)CO/N=C/c2cc(=O)[nH]c(=O)[nH]2)[nH]c(=O)[nH]1 ; O=c1cc(/C=N/OCCCO/N=C/c2ccc(O)c(O)c2)[nH]c(=O)[nH]1]

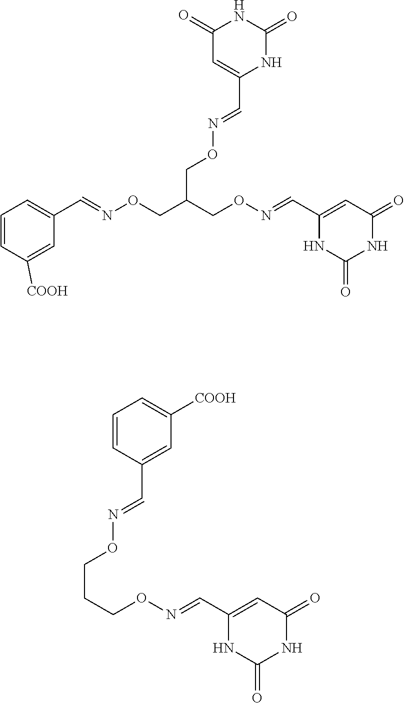 [SMILES: O=C(O)c1cccc(/C=N/OCC(CO/N=C/c2cc(=O)[nH]c(=O)[nH]2)CO/N=C/c2cc(=O)[nH]c(=O)[nH]2)c1 ; O=C(O)c1cccc(/C=N/OCCCO/N=C/c2cc(=O)[nH]c(=O)[nH]2)c1]

In some embodiments, the small molecule inhibitor of UDG is a compound of formula (III):

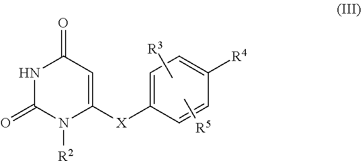

wherein
X is O, NR1, S or CH2;
R1 is hydrogen or (C1-C2)alkyl;
R2 is hydrogen; (C2-C6)alkyl optionally substituted by one or more hydroxyl, amino or carboxyl groups, or by phenyl optionally substituted by (C1-C4)alkyl, carboxyl or amino; (C1-C4)alkoxy(C2-C6)alkyl; N—(C1-C4)alkyl-carboxamido(C1-C2)alkyl; benzyl in which the phenyl moiety is optionally substituted by (C1-C4)alkyl, carboxyl or amino; pyrrolyl(C1-C2)alkyl in which the pyrrole is optionally substituted by (C1-C4)alkyl; or imidazolyl(C1-C2)alkyl in which the imidazole is optionally substituted by (C1-C4)alkyl;
R3 and R5 are the same or different and each is hydrogen; carboxamido; N—(C1-C2)alkylcarboxamido; carboxamidino; or N—(C1-C2)alkylcarboxamidino; and
R4 is (C6-C10)alkyl optionally substituted by one or more hydroxyl, amino, or carboxyl groups; (C6-C10)alkyl where the alkyl moiety can form part of a (C4-C8)cycloalkyl ring; (C6-C10)alkenyl; or (C1-C14)alkoxy; or a pharmaceutically acceptable salt thereof, with the proviso that when X is NH, R3 and R5 are hydrogen, and R4 is n-propyl, n-butyl, n-pentyl, i-pentyl, n-hexyl or n-octyl, then R2 is not hydrogen.

In some embodiments, the small molecule inhibitor of UDG is a compound of formula (IV):

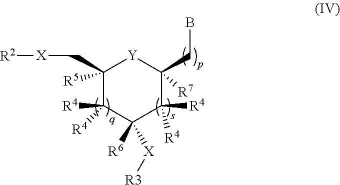

B is a nucleoside purine or pyrimidine base, or a heterocyclic analog thereof;
X are independently O, N, S or CH2;
Y represents N(R1)2, C(R)2, O, S, P, Se, B, Al or As;
R are independently a hydrogen or a lower alkyl;
R1 are independently absent or a hydrogen, or an amino protecting group;
R2 is a hydrogen, a nucleotide or oligonucleotide (e.g., 3′ linked), a phosphoryl (such as a phosphate, e.g., mono-, di- or tri-ester), a phosphonate, a phosphoramidate, a carbamate, a phosphorothioate, a phosphorodithioate, a hydroxyl blocking group, or as valence and stability permit, a halogen, a lower alkyl, a lower alkenyl, a lower alkynyl, a carbonyl (e.g., an ester, a carboxylate, or a formate), a thiocarbonyl (e.g., a thiolester, a thiolcarboxylate, or a thiolformate), a ketyl, an aldehyde, an amino, an acylarnino, an amide, an amidino, a cyano, a nitro, an azido, a sulfonyl, a sulfoxido, a sulfate, a sulfonate, a sulfamoyl, a sulfonamido, a phosphoryl, a phosphonate, a phosphinate, —(CH2)m-R8, —(CH2)m-OH, —(CH2)m-O-lower alkyl, —(CH2)m-O-lower alkenyl, —(CH2)m-O—(CH2)n-R8, —(CH2)m-SH, —(CH2)m-S-lower alkyl, —(CH2)m-S-lower alkenyl, —(CH2)m-S—(CH2)n-R8, or a solid or polymeric support;
R3 is a hydrogen, a nucleotide or oligonucleotide (e.g., 5′ linked), a phosphoryl, a phosphonate, a phosphoramidate, a carbamate, a phosphorothioate, a phosphorodithioate, a hydroxyl blocking group, or as valence and stability permit, a halogen, a lower alkyl, a lower alkenyl, a lower alkynyl, a carbonylfzc/gq an ester, a carboxylate, or, a formate), a thiocarbonyl (e.g., a thiolester, a thiolcarboxylate, or a thiolformate), a ketyl, an aldehyde, an amino, an acylamino, an amido, an amidino, a cyano, a nitro, an azido, a sulfonyl, a sulfoxido, a sulfate, a sulfonate, a sulfamoyl, a sulfonamido, a phosphoryl, a phosphonate, a phosphinate, —(CH2)m-R8, —(CH2)m-OH, —(CH2)m-O-lower alkyl, —(CH2)m-O-lower alkenyl, —(CH2)m-O—(CH2)n-R8, —(CH2)m-SH, —(CH2)m-S-lower alkyl, —(CH2)m-S-lower alkenyl, —(CH2)m-S—(CH2)n-R8, or a solid or polymeric support; and
R4, R5, R6 and R7 are each, independently for each occurrence and as valence and stability permit, hydrogen, a halogen, a lower alkyl, a lower alkenyl, a lower alkynyl, a carbonyl (e.g., an ester, a carboxylate, or a formate), a thiocarbonyl (e.g., a thiolester, a thiolcarboxylate, or a thiolformate), a ketyl, an aldehyde, an amino, an acylamino, an amido, an amidino, a cyano, a nitro, an azido, a sulfonyl, a sulfoxido, a sulfate, a sulfonate, a sulfamoyl, a sulfonamido, a phosphoryl, a phosphonate, a phosphinate, —(CH2)m-R8, —(CH2)m-OH, —(CH2)m-O-lower alkyl, —(CH2)m-O-lower alkenyl, —(CH2)m-O—(CH2)n-R8, —(CH2)m-SH, —(CH2)m-S-lower alkyl, —(CH2)m-S-lower alkenyl, —(CH2)m-S—(CH2)n-R8;
R8 is, independently for each occurrence, a substituted or unsubstituted aryl, aralkyl, cycloalkyl, cycloalkenyl or heterocycle, a carbonyl, a sulfonyl or a phosphoryl;
n and rn are independently for each occurrence zero or an integer in the range of 1 to 6;
p is zero, 1 or 2; and
q and 5 are, independently, zero, or an integer in the range of 1 to 4,
with the proviso that the sum of q and s is zero, or an integer in the range of 1 to 4, and when Y is 0, then p is 1 or 2.

By “aptamers” is meant class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996). Then after raising aptamers directed against the target of the invention as above described, the skilled man in the art can easily select those blocking or inactivating the target.

By “antibody” is meant antibody (the term including “antibody portion”) directed against the target. Said antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of the target. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the antigen may be provided as synthetic peptides corresponding to antigenic regions of interest in the target. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria, which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgGI, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgGI, IgG2, IgG3 and IgG4. In a preferred embodiment, the compound of the invention is a Human IgG4.

In another embodiment, the antibody according to the invention is a single domain antibody. The term “single domain antibody” (sdAb) or “VHH” refers to the single heavy chain variable domain of antibodies of the type that can be found in Camelid mammals, which are naturally devoid of light chains. Such VHH are also called “Nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In another aspect, the invention provides an antibody that competes for binding to the target with the antibody of the invention.

As used herein, the term “binding” in the context of the binding of an antibody to a predetermined antigen or epitope typically is a binding with an affinity corresponding to a KD of about 10−7 M or less, such as about 10−8 M or less, such as about 10−9 M or less, about 10−10 M or less, or about 10−11 M or even less when determined by for instance surface plasmon resonance (SPR) technology in a BIAcore 3000 instrument using a soluble form of the antigen as the ligand and the antibody as the analyte. BIACORE® (GE Healthcare, Piscataway, NJ) is one of a variety of surface plasmon resonance assay formats that are routinely used to epitope bin panels of monoclonal antibodies. Typically, an antibody binds to the predetermined antigen with an affinity corresponding to a KD that is at least ten-fold lower, such as at least 100-fold lower, for instance at least 1,000-fold lower, such as at least 10,000-fold lower, for instance at least 100,000-fold lower than its KD for binding to a non-specific antigen (e.g., BSA, casein), which is not identical or closely related to the predetermined antigen. When the KD of the antibody is very low (that is, the antibody has a high affinity), then the KD with which it binds the antigen is typically at least 10,000-fold lower than its KD for a non-specific antigen. An antibody is said to essentially not bind an antigen or epitope if such binding is either not detectable (using, for example, plasmon resonance (SPR) technology in a BIAcore 3000 instrument using a soluble form of the antigen as the ligand and the antibody as the analyte), or is 100 fold, 500 fold, 1000 fold or more than 1000 fold less than the binding detected by that antibody and an antigen or epitope having a different chemical structure or amino acid sequence.

Additional antibodies can be identified based on their ability to cross-compete (e.g., to competitively inhibit the binding of, in a statistically significant manner) with other antibodies of the invention in standard antigen binding assays. The ability of a test antibody to inhibit the binding of antibodies of the present invention to the target demonstrates that the test antibody can compete with that antibody for binding to the target; such an antibody may, according to non-limiting theory, bind to the same or a related (e.g., a structurally similar or spatially proximal) epitope on the target as the antibody with which it competes. Thus, another aspect of the invention provides antibodies that bind to the same antigen as, and compete with, the antibodies disclosed herein. As used herein, an antibody “competes” for binding when the competing antibody inhibits the target binding of an antibody or antigen binding fragment of the invention by more than 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% in the presence of an equimolar concentration of competing antibody.

In other embodiments the antibodies or antigen binding fragments of the invention bind to one or more epitopes of the target. In some embodiments, the epitopes to which the present antibodies or antigen binding fragments bind are linear epitopes. In other embodiments, the epitopes to which the present antibodies or antigen binding fragments bind are non-linear, conformational epitopes.

In some embodiment, the invention relates to UNG inhibitor wherein said oligonucleotide is an antisense oligonucleotide, a siRNA, a shRNA, a DNA aptamer or a RNA aptamer. Said UNG inhibitor is an UNG expression inhibitor.

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include messenger RNAs, which are modified, by processes such as capping, polyadenylation, methylation, and editing, and proteins modified by, for example, methylation, acetylation, phosphorylation, ubiquitination, SUMOylation, ADP-ribosylation, myristilation, and glycosylation.

An “inhibitor of expression” refers to a natural or synthetic compound that has a biological effect to inhibit the expression of a gene. An “inhibitor of expression” refers to any compound that has a biological effect to inhibit the expression of a target gene and/or the expression of target protein. In one embodiment of the invention, said inhibitor of expression is a short hairpin RNA (shRNA), a small inhibitory RNA (siRNA), or an antisense oligonucleotide. Preferably, the inhibitor of expression is a siRNA or a shRNA.

The target expression inhibitors for use in the present invention may be based on antisense oligonucleotide constructs. Anti-sense oligonucleotides, including anti-sense RNA molecules and anti-sense DNA molecules, would act to directly block the translation of the target mRNA by binding thereto and thus preventing protein translation or increasing mRNA degradation, thus decreasing the level of the target proteins, and thus activity, in a cell. For example, antisense oligonucleotides of at least about 15 bases and complementary to unique regions of the mRNA transcript sequence encoding the target can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as a target expression inhibitors for use in the present invention. The target gene expression can be reduced by contacting the subject or cell with a small double stranded RNA (dsRNA), or a vector or construct causing the production of a small double stranded RNA, such that the target expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, MT. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Short hairpin RNA (shRNA) or Small inhibitory RNAs (siRNAs) can function as inhibitors of gene expression for use in the invention. Gene expression can be reduced with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known.

Ribozymes can also function as target expression inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of the target mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides (ODNs) and ribozymes useful as target inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing the target. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W. H. Freeman C.O., New York, 1990) and in MURRY (“Methods in Molecular Biology,” vol. 7, Humana Press, Inc., Clifton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUCI9, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In some embodiment, the UNG inhibitor is used in combination with a PARP inhibitor.

In some embodiment, the PARP inhibitor is selected from the group consisting of olaparib, rucaparib, niraparib, talazoparib, iniparib, veliparib, Pamiparib (BGB-290), CEP 9722, E7016, E7449 and 3-Aminobenzamide.

In some embodiment, the UNG inhibitor is used in combination with cisplatin or a Pole inhibitor such as novobiocin.

In some embodiment, the UNG inhibitor is used in combination with a NMNAT1 inhibitor.

As used herein, the term “NMNAT1 inhibitor” refers to any compound selected from the group consisting of but not limited to compounds targeting Nicotinamide Nucleotide Adenylyltransferase 1. The term “NMNAT1 inhibitor” refers to compounds that bind to NMNAT1 and function as potent antagonists of NMNAT1. The term “NMNAT1 inhibitor” has its general meaning in the art and refers to a compound that selectively inactivates NMNAT1. Typically, a NMNAT1 inhibitor is a small organic molecule, a polypeptide, an aptamer, an oligonucleotide (antisense oligonucleotides, siRNA, shRNA, DNA and RNA aptamers), or an antibody. NMNAT1 inhibitors are well-known in the art as such as described in WO2013/130672; Kusumanchi et al., 2013.

The term “NMNAT1 inhibitor” refers to any compound selected from but not limited to siRNA such s34981 (hNMNAT1 siRNA) (Kusumanchi et al., 2013; WO2013/130672), siRNA ID130437, siRNA ID130438, siRNA ID130439, siRNA ID212976, siRNA ID212977 (Thermo Fisher Scientific).

In some embodiment, said NMNAT1 inhibitor is an antisense oligonucleotide, a siRNA, a shRNA, a DNA aptamer or a RNA aptamer.

In some embodiment, the UNG inhibitor is used in combination with a SIRT6 inhibitor.

In some embodiment, said SIRT6 inhibitor is selected from the group consisting of OSS128167, Trichostatin A and SIRT6 inhibitory Quercetin derivatives luteolin, catechin gallate and gallocatechin gallate.

### Pharmaceutical Composition

In a further aspect, the invention relates to a pharmaceutical composition comprising a Uracil DNA glycosylase (UNG) inhibitor and a pharmaceutical acceptable carrier for use in the treatment of resistant Homologous Recombination Deficiency (HRD) cancer in a subject in need thereof, wherein the UNG inhibitor is a molecule capable of silencing the gene expressing the UNG enzyme or a molecule inhibiting UNG enzymatic properties.

Also part of the invention is a pharmaceutical composition comprising (i) a Uracil DNA glycosylase (UNG) inhibitor, which is a molecule capable of silencing the gene expressing the UNG enzyme or a molecule inhibiting UNG enzymatic properties, (ii) at least one of a PARP inhibitor, cisplatin, a Pole inhibitor, a NMNAT1 inhibitor, and a SIRT6 inhibitor, as described above, and (iii) and a pharmaceutical acceptable carrier.

In some embodiment, the resistant HRD cancer is resistant BRCA-associated cancer, chemo-resistant HRD cancer or chemo-resistant BRCA-associated cancer.

Typically, the compound of the invention may be combined with pharmaceutically acceptable excipients, and optionally sustained-release matrices, such as biodegradable polymers, to form therapeutic compositions.

Typically, the compounds according to the invention as described above are administered to the subject in a therapeutically effective amount.

By a “therapeutically effective amount” of the compound of the present invention as above described is meant a sufficient amount of the compound at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the compound of the present invention for the symptomatic adjustment of the dosage to the patient to be treated. A medicament typically contains from about 0.01 mg to about 500 mg of the compound of the present invention, preferably from 1 mg to about 100 mg of the compound of the present invention. An effective amount of the drug is ordinarily supplied at a dosage level from 0.0002 mg/kg to about 20 mg/kg of body weight per day, especially from about 0.001 mg/kg to 7 mg/kg of body weight per day.

In a particular embodiment, the compound according to the invention may be used in a concentration between 0.01 μM and 20 μM, particularly, the compound of the invention may be used in a concentration of 0.01, 0.05, 0.1, 0.5, 1.0, 2.5, 5.0, 10.0, 15.0, 20.0 μM.

According to the invention, the compound of the present invention is administered to the subject in the form of a pharmaceutical composition. Typically, the compound of the present invention may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The compound of the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized agents of the present inventions into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the compound of the present invention plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

Pharmaceutical compositions of the invention may include any further compound which is used in the treatment of cancer such as described above. These compounds include in particular a PARP inhibitor, cisplatin, a Pole inhibitor, a NMNAT1 inhibitor, or a SIRT6 inhibitor, as described above. Preferably, the pharmaceutical composition comprises a UNG inhibitor and a PARP inhibitor or a Pole inhibitor, as described above.

In some embodiments, the pharmaceutical compositions of the invention may include any further compound which is used in the treatment of HRD cancer, BRCA-associated cancer, resistant HRD cancer or resistant BRCA-associated cancer.

In one embodiment, said additional active compounds may be contained in the same composition or administrated separately.

In another embodiment, the pharmaceutical composition of the invention relates to combined preparation for simultaneous, separate or sequential use in the treatment of resistant HRD cancer in a subject in need thereof.

In some embodiments, the pharmaceutical composition of the invention relates to combined preparation for simultaneous, separate or sequential use in the treatment of resistant BRCA-associated cancer in a subject in need thereof.

In some embodiments, the pharmaceutical composition of the invention relates to combined preparation for simultaneous, separate or sequential use in the treatment of chemo-resistant HRD cancer in a subject in need thereof.

In some embodiments, the pharmaceutical composition of the invention relates to combined preparation for simultaneous, separate or sequential use in the treatment of chemo-resistant BRCA-associated cancer in a subject in need thereof.

### Treatment

In a further aspect, the invention relates to a method of treating resistant Homologous Recombination Deficiency (HRD) cancer such as resistant BRCA-associated cancer, chemo-resistant HRD cancer or chemo-resistant BRCA-associated cancer in a subject in need thereof, comprising administering to the subject a therapeutically effective amount of a Uracil DNA glycosylase (UNG) inhibitor.

The method of treating of the invention may include further administration of a PARP inhibitor, cisplatin, a Pole inhibitor, a NMNAT1 inhibitor, or a SIRT6 inhibitor, as described above. Typically, the administration of the compound of the invention may be combined with administration of other active compound, pharmaceutically acceptable excipients, and optionally sustained-release matrices, such as biodegradable polymers. Typically, the additional active compounds as described above are administered to the subject in a therapeutically effective amount.

In one embodiment, said additional compounds may be contained in the same composition or administrated separately.

The invention is further illustrated by the following figures and examples.

### FIGURES

FIG. 1. a. Quantification and representative images of clonogenic formation of wild type (WT) and BRCA2−/− (clones C1 and C2) RPE-1 cells with and without BRCA2 cDNA complementation, after transfection with the indicated NMNAT1 siRNAs. b. Clonogenic formation and WB-analysis of BRCA2 mAID HeLa cells in the presence or absence of auxin (IAA) after transduction with viral particles carrying NMNAT1 shRNA or control. c. Clonogenic formation of WT and BRCA1−/− RPE-1 cells after transfection with siNMNAT1 #2. d. Clonogenic formation of WT and NMNAT1−/− RPE-1 cells after transfection with the indicated BRCA1/2 siRNAs. e. Quantification and representative images of clonogenic formation of the indicated HR-proficient and HR-deficient cells upon siNMNAT1. f. DNA damage quantification by alkaline COMET assay of P53−/− RPE-1 cells in the presence or absence of NMNAT1. g. Survival assay of WT and NMNAT1−/− RPE-1 cells exposed to the indicated doses of the PARP inhibitor rucaparib (PARPi).

FIG. 2. a,b. Proliferation curve of WT and NMNAT1^{-/-} cells in P53^{-/-} (a) and P53^{+/+} (b) RPE-1. c. DNA damage quantification by alkaline COMET assay of the same cells as in a,b.

FIG. 3. a. WB analysis showing PARP1 activation in WT and NMNAT1^{-/-} RPE-1 cells (G9) complemented with wild type (WT), catalytically dead (MUT, W169A) NMNAT1 cDNA or empty vector (EV) following exposure to PARG inhibitor and MMS. b. DNA damage quantification by alkaline COMET assay of WT and NMNAT1^{-/-} (clone G9) RPE-1 cells complemented or not (EV) with wild type (WT) or catalytically dead (MUT, W169A) NMNAT1 cDNA. c.d, Clonogenic formation of cells as in b in response to PARPi (c) or following transfection with either BRCA2 or control (siCTRL) siRNAs (d left panel). Representative images are shown in d right panel.

FIG. 4: a, Schematic of the drug-resistant cell lines used in this work. b, Clonogenic formation of wild type (WT), BRCA2^{-/-} (clones C1 and C2) and BRCA2^{-/-}-derived PARPi-resistant RPE-1 cells after control or NMNAT1 (siNMNAT1 #2) siRNAs transfection. c, Clonogenic formation of parental and derived PARPi-resistant CAPAN-1 cells after transduction with shNMNAT1 lentiviral particles. Representative images are shown in the right panel. d, Clonogenic formation of PEO-1 and PEO-4 after transduction with shNMNAT1 lentiviral particles. Lentiviral particles carrying scramble shRNA (shSCR) were used as control for all the experiments shown in c,d.

FIG. 5. a. Survival assay of RPE-1 cells in response to PARPi after transfection with siRNAs targeting the indicated sirtuins. b,c, Quantification of clonogenic assay of WT and SIRT6−/− RPE-1 cells in response to PARPi (b) or after transfection with siBRCA2 or control (siCTRL) (c). Representative images are shown in the lower panel.

FIG. 6. a, Schematic of the drug-resistant cell lines used in this work. b, Clonogenic formation of parental and derived PARPi-resistant CAPAN-1 cells after transduction with shSIRT6 lentiviral particles. c, Clonogenic formation of PEO-1 and PEO-4 after transduction with shSIRT6 lentiviral particles. d, Clonogenic formation of parental and derived cisplatin-resistant CAPAN-1 cells after transduction with shSIRT6 viral particles. Lentiviral particles carrying scramble shRNA (shSCR) were used as control for all the experiments shown in b,c,d.

FIG. 7. a, Clonogenic formation of wild type (WT) and BRCA2−/− (clones C1 and C2) following exposure to the indicated doses of SIRT6 inhibitor OSS_128167. b, Clonogenic formation of wild type (WT), BRCA2−/− (clones C1 and C2) and BRCA2−/−-derived PARPi-resistant RPE-1 cells following exposure to 0.5 mM SIRT6 inhibitor OSS_128167. c, Clonogenic formation of parental and derived PARPi-resistant CAPAN-1 clones following exposure to the indicated doses of SIRT6 inhibitor OSS_128167. The normal breast epithelial cells MCF10A were included as an HR-proficient cell line.

FIG. 8. Waterfall plot (a) of the PARPi-resistant BRCA1-mutated PDX HBCx-11 at day 32 upon treatment with either the PARP inhibitor olaparib (PARPi) or in combination with the SIRT6 inhibitor OSS_128167 (SIRT6i). The number of tumors classified either as stable or progressive disease are plotted on the bottom panel (b).

FIG. 9. UNG functions downstream of SIRT6 in the survival of BRCA1/2-mutated tumors. a. Scatter plot of the SILAC forward (FWD) and reverse (REV) proteomic experiments showing that UNG is significantly downregulated in the nuclei of SIRT6 knockout cells (SIRT6−/−). b. Immunoblot validation of the MS-proteomics data showing UNG decreased protein level in SIRT6−/− cells. c. Survival clonogenic assay in wild type (WT) and BRCA2 knockout RPE_1 clones (cl.1 and cl.2) upon depletion of UNG protein. UNG protein was downregulated by two independent siRNA sequences (si #6 and si #7). d. Survival clonogenic assay in parental (PARPi-naive) and PARPi-resistant OVCAR8 cells. UNG protein was downregulated by two independent siRNA sequences (si #6 and si #7). OVCAR8 is a BRCA1-hypermethylated ovarian tumor cell line. e,f. Quantification of ssDNA gaps by DNA fibre assay in cells depleted for either SIRT6 (e) or UNG (f).

FIG. 10. UDG depletion kills PARPi-naive and -resistant HRD cells. a. Survival assay of WT and UNG depleted cells upon exposure to the indicated doses of the PARP inhibitor (PARPi) rucaparib. b. Quantification of clonogenic formation of WT and two BRCA2−/− clones (C1 and C2) upon depletion of UNG by two different siRNA (#6 and #7). c. Quantification of clonogenic formation of WT and UNG−/− clone upon depletion of BRCA2 by siRNA. A western blot shows UNG knockout efficiency. d. Quantification of clonogenic formation of PARPi naive and resistant BRCA1- and BRCA2-mutated cancer cell lines (OVCAR8 and CAPAN-1), upon depletion of UNG by two different siRNA (#6 and #7).

FIG. 11. Uracil excision is regulated by nuclear NAD⁺. a. Quantification of post-replicative ssDNA gaps using DNA fiber assay in P53^{-/-} RPE-1 cells after 48-hours transfection with the indicated siRNAs. b. Schematic of 2′-deoxyuridine (dUTP)/thymidine (dTTP) biosynthetic pathway. The biological processes involved are in italic. c. Nuclear fractionation and immunoblot profiling of the indicated proteins following 24-hour drug exposure (0.1 μM FdUrd+5 μM TAS-114) of WT and UNG^{-/-} cells. For the washed-out samples (w.o.), cells were harvested 24 hours after drug removal. Western blot images are representative of three experiments. d. Left, quantification of DNA breaks (left, − rUNG) and gDNA-uracil (right, + rUNG) in wild type (WT) and NMNAT1^{-/-} cells upon 24-hour FdUrd treatment (20 μM) and the indicated hours after drug washout (w.o.). UNG^{-/-} cells before and after treatment with rUNG are also shown. Right, representative images of the cells treated as in (left). e. Top, Schematic of the POND workflow. Left, Immunoblot analysis before (input) and after capture of EdU-labeled DNA in WT and NMNAT1^{-/-} cells. No EdU pulse (EdU−) and thymidine chase (Thym+) were used as control of specificity and RF proximity, respectively. Right, Quantification of UNG protein level captured as in (left) normalized over captured PCNA and H3. f. Quantification of γH2AX foci formation before and after 24 h treatment of WT (grey) and NMNAT1^{-/-} (orange) cells with the indicated doses of TAS-114 (μM). g. Quantification (left) and representative images (right) of 12-day clonogenic survival assay following transfection with siRNAs and drug treatment of WT and NMNAT1^{-/-} cells as indicated. 48 hours after siRNA transfection, 5 μM TAS-114 was used or not in combination with the indicated dosed of FdUrd for 24 hours before drug washout.

FIG. 12. UNG inhibition leads to SMUG1-mediated DNA damage and HR activation.

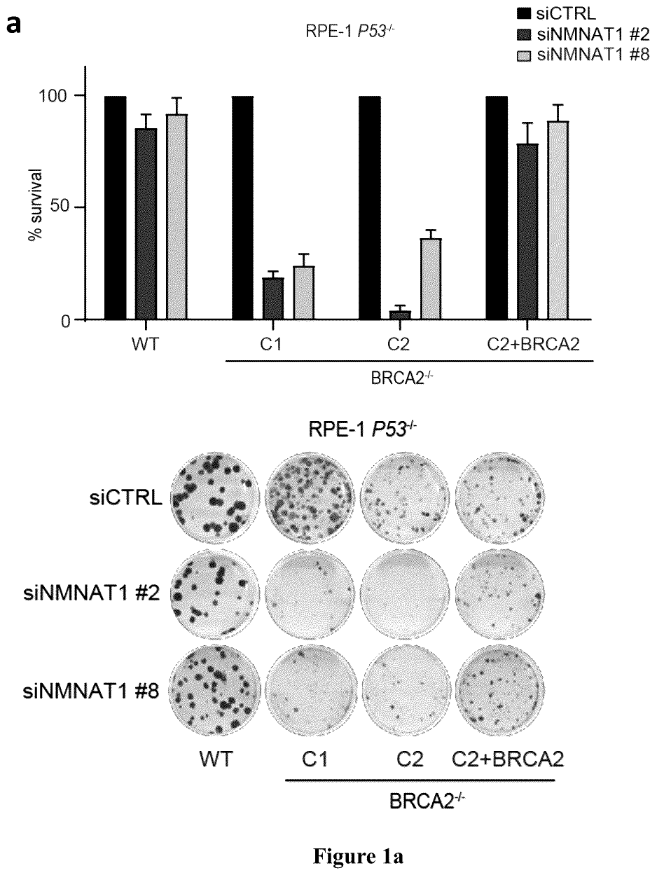

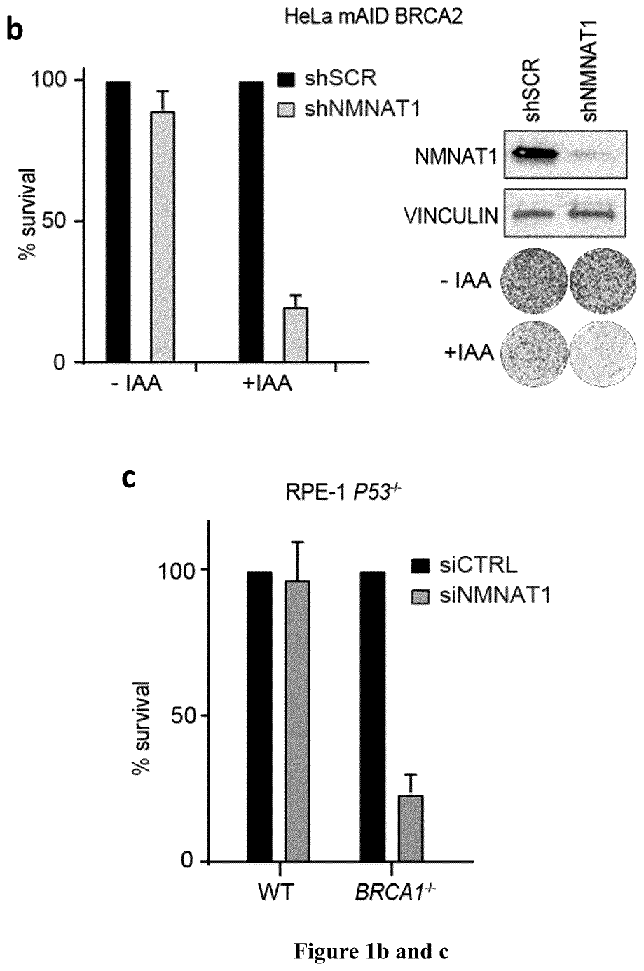

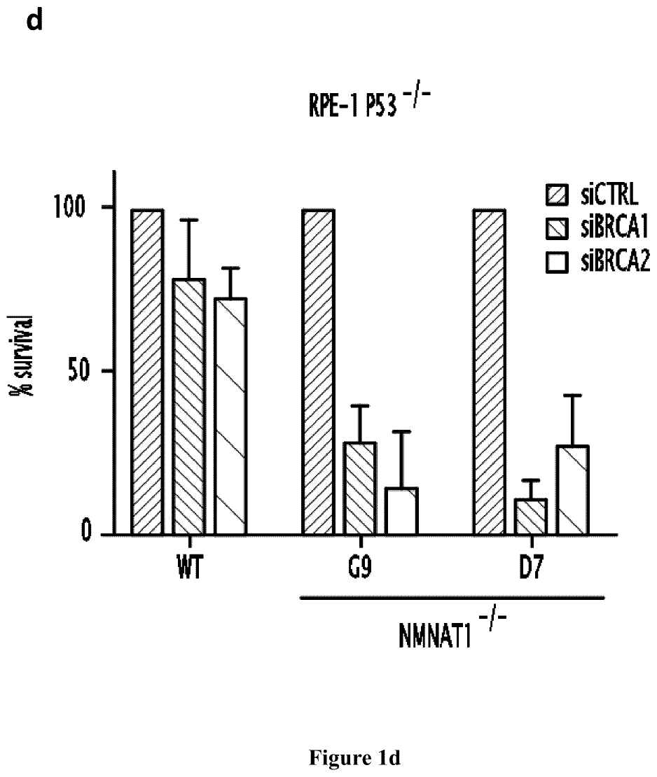

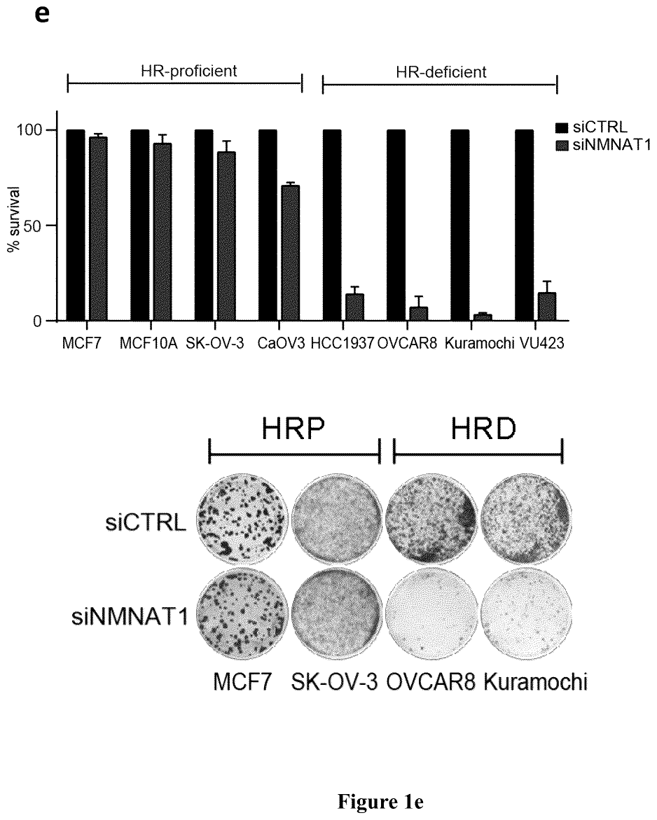

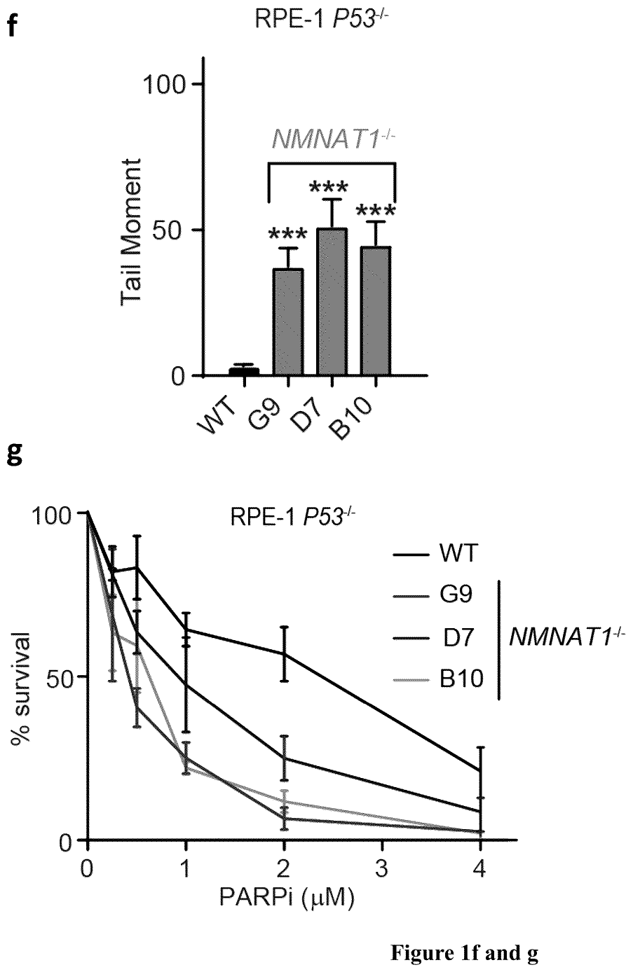

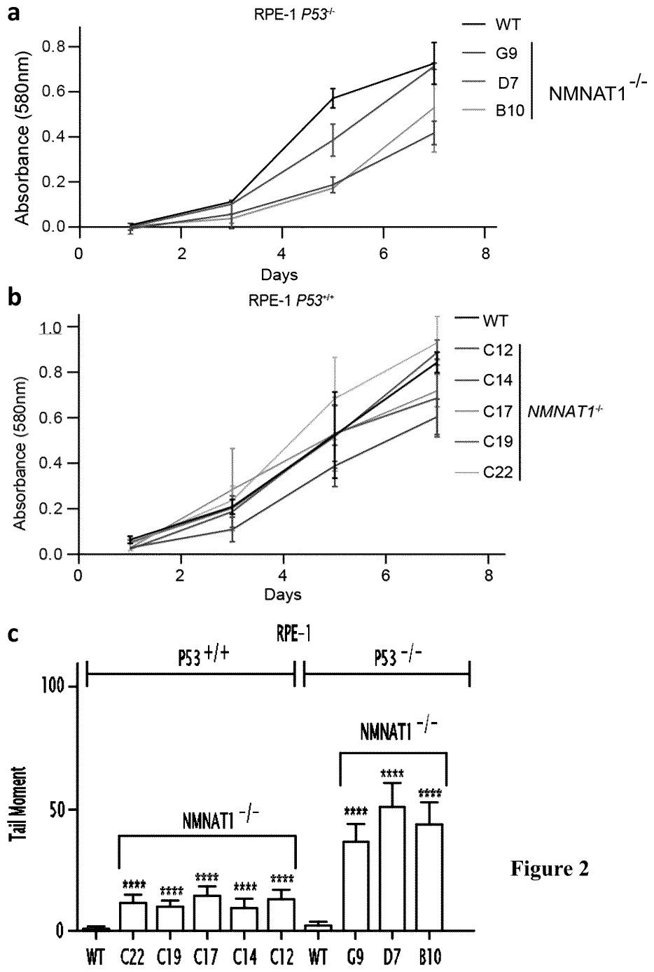

a. Quantification (left) and representative images (right) of the cell cycle profile of WT and UNG−/− cells treated or not as in (FIG. 1c). Quantification (left) is from the gates highlighted in red (right). b. Nuclear fractionation and immunoblot profiling of the indicated proteins following drug exposure of WT and UNG−/− cells as in (FIG. 1c). Western blot images are representative of three experiments and protein lysates are the same as shown in (FIG. 1c). c. immunoblot profiling of HMCES DNA adducts following RADAR in WT and UNG−/− cells treated as in (FIG. 2b). MG132 10 μM was added or not 3 hours before cell collection. d. Immunofluorescence-based quantification (top) and representative images (bottom, left) of SMUG1 (left), HMCES (center) and γH2AX (right) foci in WT and UNG−/− cells treated as in in (FIG. 2b). Bottom right, Quantification of HMCES/γH2AX colocalizing foci. e. Immunoblot profiling of the indicated proteins in the chromatin fraction of UNG−/− cells following siRNA transfection and drug treatment as indicated. Cells were treated or not with the indicated drugs (0.1 μM FdUrd+5 μM TAS-114 for 24 hours) 48 hours after siRNA transfection. f. Quantification of the 12-day clonogenic survival assay in P53−/− RPE-1 cells transfected with the indicated siRNAs and 48-hour later continuously exposed or not to the indicated doses of rucaparib (PARPi). g. Immunofluorescence-based quantification (left) and representative images (right) of RAD51 foci in WT (left) and UNG−/− (right) cells treated as in (FIG. 2b) A representative image of γH2AX (top) and SMUG1 (bottom) foci is also shown.

FIG. 13. dU misincorporation results in under-replicated DNA and chromosomal fragmentation in HR-deficient cells.

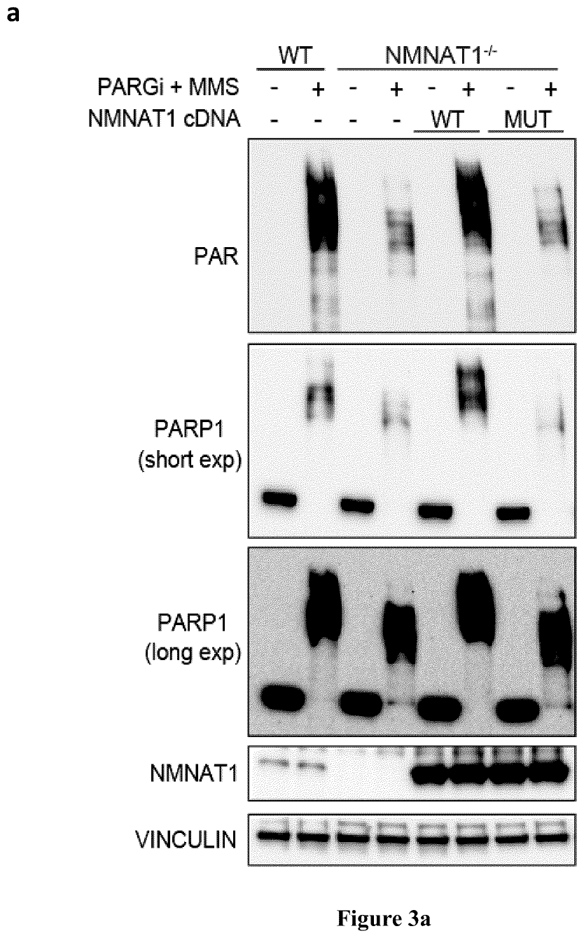

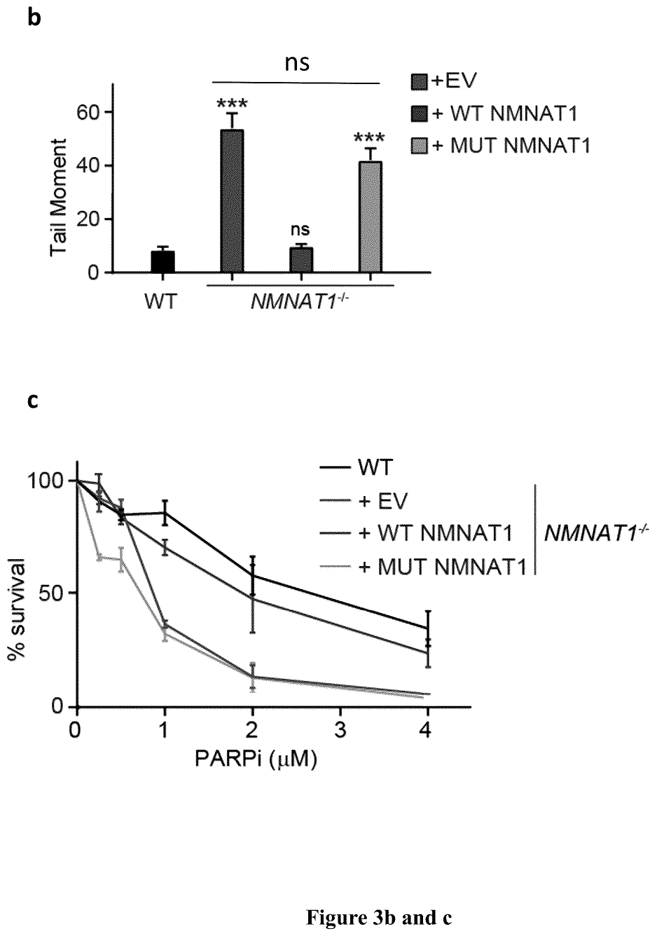

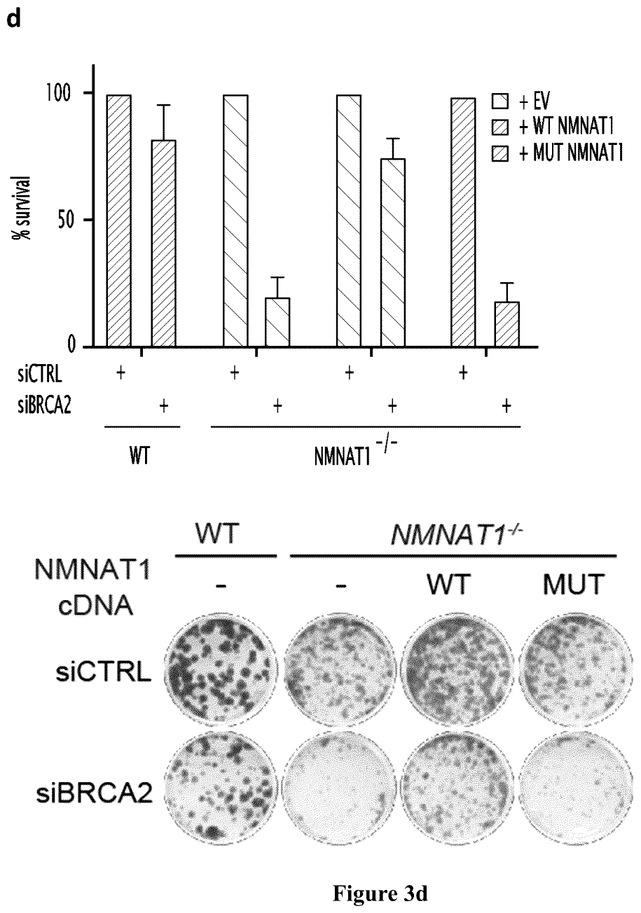

a. representative images of pulverized/fragmented chromosomes and chromosomal translocations in BRCA2^{-/-} cells treated with 0.1 μM FdUrd+5 μM TAS-114 for 24 hours and analyzed 48 hours after drug washout (n=3). b. Quantification (left) and representative images (right) of anaphase ultrafine bridges (UFBs) in BRCA2^{-/-} cells upon treatment as in (FIG. 3a) and analyzed upon 24-hour drug washout (n=3). c. Quantification (left) and representative images (right) of 53BP1 NBs (top) and micronuclei (bottom) in BRCA2^{-/-} cells upon treatment as in (FIG. 3a) and analyzed upon 48-hour drug washout (n=4). d. Quantification of the 12-day clonogenic survival assays in BRCA2^{-/-} cells transfected with indicated siRNAs and 48-hour later treated or not with 1 μM TAS-114 for the duration of the experiment.

FIG. 14. Targeting uracil excision by UNG kills HR-deficient tumors, regardless of their resistance to PARPi.

a. Bottom, quantification of the 12-day clonogenic survival assay in WT and two BRCA2^{-/-} clones (C1 and C2) following transfection with the indicated siRNAs. Top, immunoblot showing UNG depletion. The arrows indicate UNG1 (lower) and UNG2 (upper) (n=3). b. Quantification (bottom) and representative images (top) of the 12-day clonogenic survival assay in BRCA2^{-/-} cells following transfection with the indicated siRNA. c. Quantification of the 12-day clonogenic survival assay in the indicated PARPi-resistant HR-deficient cell lines upon transfection with the indicated siRNA.

FIG. 15. UNG and SMUG1 interplay in uracil excision is controlled by nuclear NAD⁺.

a. Top, Immunoblot profiling of WT and NMNAT1^{-/-} RPE-1 cells for the indicated proteins. Bands corresponding to UNG1 (lower MW) and UNG2 (higher MW) are indicated. The star (*) indicates an unspecific band. Cells were treated for 24 hours with the indicated doses of FdUrd, with and without 5 μM TAS-114. Bottom, Cell cycle distribution of WT (+/+) and NMNAT1^{-/-} (−/−) RPE-1 cells treated as in (top) (n=2). b. Quantification of gDNA-uracil and DNA breaks by modified comet assay with (right) and without (left) recombinant UNG (rUNG) respectively, in WT RPE-1 cells upon 24-hour FdUrd treatment with and without 5 μM TAS-114. Results are shown as tail moment. At least 80 comets per condition was measured using CometScore. Results shown are mean±standard error of the mean (SEM) (n=3).c, Top, NAD⁺ salvage pathway and role of NMNAT1 (in red) in the control of nuclear NAD⁺ homeostasis. The enzymes involved are indicated in bold and italic. Bottom, Immunoblot profiling of NMNAT1, ADP-ribosylation (PAR) and PARP1 (short and long exposures) in WT and NMNAT1^{-/-} cells complemented or not with wild type (WT) or catalytically-dead mutant (MUT) NMNAT1 (W169A). PARP1 was detected after membrane stripping of the anti-ADP ribosylation antibody. Cells were treated or not with PDD 00017273 (PARGi, 10 μM) for 2 hours and 10 mM MMS was added the last 10 minutes before cell lysis. Vinculin was used as loading control. d. Quantification of the 12-day clonogenic survival assay of the same cells as in (c, bottom) treated or not with the indicated doses of rucaparib (PARPi) for the duration of the experiment (n=3). e. Quantification of the 12-day clonogenic survival assay in WT (black) and NMNAT1^{-/-} (red) cells following exposure to the indicated doses of the indicated DNA-damaging drugs (n=3). f. Quantification of the 12-day clonogenic survival assay in WT, NMNAT1^{-/-}, and UNG^{-/-} cells following 24-hour exposure to the indicated doses of FdUrd with or without 5 μM TAS-114 (n=3). g. Quantification of the 12-day clonogenic survival assay in WT (black), NMNAT1^{-/-}, and UNG^{-/-} cells following 24-hour exposure to the indicated doses of TAS-114. (n=3). For WT cells, data are the same as in (FIG. 14c). h. Top, RT-qPCR quantification of DUT mRNA levels following 72-hour transfection of WT RPE-1 cells with DUT siRNA (siDUT) or relative control (siCTRL). Results are represented as fold change over siCTRL. (Bottom), Quantification of the 12-day clonogenic survival assay in WT (left), NMNAT1^{-/-} (middle), and UNG^{-/-} (right) cells following transfection with DUT siRNA and relative control (siCTRL). Data were expressed as a percentage (mean±s.e.m.) of colonies formed in each cell line relative to the appropriate siCTRL (n=3). i. Quantification of gDNA-uracil and DNA breaks as in (b) in WT and NMNAT1^{-/-} cells upon 0.08 μM FdUrd treatment with and without 5 μM TAS-114 for 24 hours.

FIG. 16. SMUG1 creates DSBs during replication in the absence of NMNAT1/UNG-mediated uracil excision.

a. Cell cycle distribution of WT (+/+) and NMNAT1^{-/-} (−/−) RPE-1 cells upon 10 μM FdrU for 24 hours, and drug washout (w.o.) for 24 and 48 hours. Data are represented as percentage of cells in the different cell cycle phases (n=2). b. Mass spectrometry-based quantification of the indicated metabolites (n=3). c. Quantification of number of γH2AX foci per nucleus in WT and UNG^{-/-} RPE-1 cells after exposure or not to TAS-114, as quantified by immunofluorescence. Foci were scored separately in cells that are in S-phase (EdU positive, right) or other cell cycle phases (EdU negative, left). At least fifty EdU-positive and fifty EdU-negative cells were scored (n=3). d. Quantification of number of γH2AX foci per nucleus in WT, NMNAT1^{-/-} and UNG^{-/-} RPE-1 cells after exposure or not to 5 μM FdUrd for 24 hours followed or not by drug washout (w.o.) for the indicated durations (n=3). e. Immunoblot profiling of the indicated proteins after nuclear separation of WT and NMNAT1^{-/-} cells into soluble (nucleosol) and insoluble (chromatin) fractions following washout (w.o. in hours) after 24 hours drug exposure. f. Immunoblot profiling of SMUG1, TDG and MBD4 in WT RPE-1 cells 72 hours after transfection with the indicated siRNA pools. Vinculin was used as loading control.

FIG. 17. Overcoming UNG excision capability results in SMUG1-HMCES trapping on chromatin, sustained PARP1 hyperactivation and HR activation.

a. Immunoblot of HMCES and histone H3 before (input) and after capture using iPOND technique. All the samples (WT and UNG^{-/-} cells) were treated with 0.001 μM FdrU and 5 μM TAS-114 for 24 hours followed or not by 24-hour drug washout (w.o.). After treatment, cells were pulsed with EdU for 10 minutes and—where indicated—followed by thymidine (Thym) chase for an additional 30 minutes. EdU was omitted in the negative control. The protein samples before capture (input) represent the 0.13% of the material used for the EdU-labelled DNA pulldown (capture) (n=2). b. Immunofluorescence-based quantification of the colocalization between the indicated protein foci upon 24-hour combined treatment of 5 μM TAS-114 and 0.1 μM FdrU with and without 24-hour drug washout (w.o.) in WT and UNG^{-/-} cells. (Left), Percentages of colocalizing foci between SMUG1 and HMCES (left), SMUG1 and γH2AX. (middle) and HMCES and SMUG1 (right) are shown, respectively.

FIG. 18. dU misincorporation results in under-replicated DNA and chromosomal fragmentation in HR-deficient cells.

a. Immunoblot profiling of BRCA2 protein in WT and BRCA2^{-/-} RPE-1 cells (clones C1 and C2). Clone C2 was used to perform BRCA2 complementation with wild-type BRCA2 cDNA. Vinculin was used as loading control. b. Immunofluorescence-based quantification of ionizing radiation (IR)-induced RAD51 foci in WT and BRCA2^{-/-} clones. Cells were irradiated with 8 Gy and fixed and stained 6 hours after irradiation (n=3).

FIG. 19. Synthetic lethality between NMNAT1/UNG inhibition and BRCA1/2 mutations.

a. Quantification (top) and representative image (bottom right) of the 12-day clonogenic survival assay in WT and BRCA2^{-/-} cells following transduction with viral particles carrying either pLentiCRISPRv2 Neo_UNG gRNA (UNG gRNA) or relative control (CTRL gRNA) (n=3). Cells were seeded for clonogenic assay after selection with G418. Bottom left, Immunoblot analysis of UNG after G418 selection. b. Left, quantification of the 12-day clonogenic survival assay in WT and BRCA2^{-/-} RPE-1 cells following transduction with retroviral particles carrying either UGI or relative control (EV) (n=3). Right, qPCR-based quantification of UNG activity in the same cell lines as in (left). c. Left, Quantification of the 12-day clonogenic survival assay in WT and UNG^{-/-} cells following transfection with siBRCA2 (n=3). Right, immunoblot analysis of UNG in the same samples as in (left). The arrows indicate UNG1 (lower) and UNG2 (upper). Vinculin was used as a loading control. d. Quantification of the 12-day clonogenic survival assay in WT and NMNAT1^{-/-} clones (G9 and D7) following transfection with the indicated siRNAs (n=3). e. Left, Quantification (left) and representative images (right) of the 12-day clonogenic survival assay in WT and NMNAT1^{-/-} cells complemented or not (EV, empty vector) with wild-type (WT) or catalytically-dead mutant (MUT:W169A) NMNAT1 cDNA following transfection with siBRCA2 (n=3). f. Quantification of the 12-day clonogenic survival assay in the HR-deficient cancer cell lines CAPAN1 (BRCA2 mutated) and OVCAR8 (BRCA1 promoter hypermethylation) following transfection with siUNG #7 (n=3). g. Quantification (left) and representative images (right) of the 12-day clonogenic survival assay in a panel of HR-proficient and HR-deficient cancer and normal (MCF10A) cell lines following transfection with siNMNAT1 (n=3).

FIG. 20. Targeting UNG-mediated uracil excision kills a subset of PARPi-resistant HR-deficient tumors.

a. Quantification of the 12-day clonogenic survival assay of WT, PARPi-naive (C1 and C2) and -resistant (C1-3, C1-5, C2-1, and C2-2) BRCA2^{-/-} RPE-1 cells continuously exposed to the indicated doses of rucaparib (PARPi) (n=3). b. Replication fork degradation assay by DNA combing. WT, PARP-naive (C2) and -resistant BRCA2^{-/-} RPE-1 clones (C2-1 and C2-3) were labelled consecutively with the thymidine analogs IdU and CIdU for 30 minutes followed by 3 hours of 4 mM hydroxyurea treatment to stall the replication forks. The CIdU/IdU ratio was used as a measure of replication fork degradation (n=3). c. Quantification of the 12-day clonogenic survival assay of PARPi-naive parental CAPAN-1 cells and the derived PARPi-resistant clones. All the cell lines were continuously exposed to the indicated doses of rucaparib during the assay (n=3). d. Quantification of the RAD51 foci per nuclei in cells as in (c) 6 hours after exposure (IR) or not (UT) to 8 Gy of ionizing radiation (n=2). e. Quantification of the 12-day clonogenic survival assay of WT, PARPi-naive (C1 and C2) and -resistant (C1-3, C1-5, C2-1, and C2-2) BRCA2^{-/-} RPE-1 clones following knockdown of NMNAT1 (siNMNAT1). The survival percentage of each cell line is normalized to the relative control (siCTRL) (n=3). f. Quantification (left) and representative images (right) of the 12-day clonogenic survival assay of the breast normal MCF10A (HR-proficient) and the indicated CAPAN1 cell lines, including the parental (PARPi naive) and PARPi resistant derived clones (C1-C17) following transduction with either shRNA targeting NMNAT1 (shNMNAT1) or non-targeting shRNA (shSCR) carrying particles. The survival percentage of each cell line is normalized to the relative control (shSCR) (n=3). Representative images of PARPi response (PARPi) and the relative control (DMSO) of each cell line is also shown. g. Survival curves for the relative tumor volume (RTV) of the subcutaneous OVCAR8-xenografts in nude mice. Mice carrying the parental (black) or the PARPi-resistant (red) OVCAR8-xenografts were treated twice a week with rucaparib (PARPi, dashed lines) or the vehicle (continuous line). h. Left, growth of PARPi-resistant OVCAR8 (BRCA1-deficient) xenografts in vivo. Top, immunoblot showing efficiency of NMNAT1 silencing. Right, relative tumor volumes (RTV) for individual mice treated as in (left) after five weeks of growth in the presence or absence of doxycycline (doxy). i. Overall survival for mice treated with vehicle or doxycycline as in (h).

### EXAMPLES

### Example 1: NMNAT1 Inhibition Kills BRCA1 and BRCA2-Mutated Tumor Cells

To study the role of NMNAT1-produced nuclear NAD+ in homologous recombination-deficient (HRD) cells, the inventors generated several BRCA1/2 isogenic cell systems, namely BRCA1−/− and BRCA2−/− knockout clones in P53−/− RPE-1 cells and BRCA2 mini auxin-inducible degron (mAID) in HeLa cells. In P53−/− RPE-1 cells, NMNAT1 knockdown by two different short interfering RNA sequences (siRNA) impaired the clonogenic ability of BRCA2−/− clones, while having no effect on the survival of parental HRP cells (FIG. 1a). Complementation of BRCA2−/− cells with a full-length BRCA2 cDNA (C2+BRCA2) rescued the survival of BRCA2−/− cells upon NMNAT1 depletion (FIG. 1a). Likewise, in the mAID BRCA2 HeLa isogenic model NMNAT1 knockdown by short hairpin RNA (shRNA) reduced cell growth only in the presence of auxin, i.e. upon BRCA2 protein degradation (FIG. 1b). Furthermore, NMNAT1 silencing also impaired the clonogenic ability of BRCA1−/− P53−/− RPE-1 cells, while sparing parental HRP cells (FIG. 1c). These data suggest that NMNAT1 is synthetically lethal with BRCA1/2.

To corroborate these findings, the inventors used a complementary approach by silencing BRCA1/2 in NMNAT1−/− RPE-1 cells that were generated by CRISP-Cas9 genome editing. While having only a mild effect on P53−/− RPE-1 cells, knockdown of BRCA1/2 by siRNA completely impaired the clonogenic ability of NMNAT1−/− P53−/− RPE-1 cells (FIG. 1d).

Next, the inventors compared siNMNAT1 cytotoxicity between HRP and HR-deficient (HRD) cancer cells. siNMNAT1 had a strong impact on the clonogenic ability of HRD cells, whereas it induced only mild effects in HRP cells, including the immortalized normal breast epithelial MCF-10A cells (FIG. 1e). Altogether, the data show that NMNAT1 is synthetically lethal with BRCA1/2.

The inventors quantified DNA breaks in RPE-1 cells with and without NMNAT1 by alkaline COMET assay. P53−/− NMNAT1−/− clones showed higher amount of DNA lesions if compared with parental cells (FIG. 1f), indicating that NMNAT1 contributes to maintain genome stability.

Accumulation of DNA lesions can result in increased sensitivity to genotoxic drugs. Given that NMNAT1 functions upstream of PARP1 by providing NAD+, the reduced PARP1 activity upon NMNAT1 loss might account for the observed synthetic lethality between NMNAT1 and BRCA. Interestingly, NMNAT1−/− cells were sensitized to the PARP inhibitor rucaparib (PARPi) (FIG. 1g), suggesting that the role of NMNAT1 is not completely epistatic with PARP1, but rather nuclear NAD+ could be used by other downstream enzymes to sustain HRD cell survival.

### NMNAT1 Loss is not Detrimental in Normal Immortalized P53+/+ Cells

To rule out any possible detrimental effect of NMNAT1 loss in HRP cells, the inventors monitored the proliferation rate of P53−/− NMNAT1−/− RPE-1 and parental cells for one week. All the three NMNAT1−/− clones showed a mild reduction in terms of proliferation when compared to parental P53−/− RPE-1 cells (FIG. 2a). To better evaluate the effect of NMNAT1 loss in normal tissues, the inventors generated NMNAT1 knockout clones in P53+/+ RPE-1, as normal cells are P53-proficient. Contrarily, when P53+/+ NMNAT1−/− RPE-1 clones were compared to parental cells, the inventors observed that NMNAT1 loss did not have any impact in terms of cell proliferation, as shown by the similar growth of P53+/+ NMNAT1−/− clones (C12, C14, C17 C19 and C22) and parental P53+/+ cells (FIG. 2b). These data indicate that whereas NMNAT1 loss slightly reduces the proliferation of P53−/− cells, it does not have any detrimental effect in P53+/+ cells, thus suggesting that targeting NMNAT1 could have no toxic impact in normal tissue.

The observed accumulation of DNA lesions in NMNAT1−/− P53−/− cells (FIG. 1g) might account for the reduced proliferation upon NMNAT1 loss in P53−/− RPE-1 cells (FIG. 2a). However, when the inventors measured DNA breaks by alkaline COMET assay in NMNAT1−/− P53+/+ cells, the increase of DNA breaks was less considerable than in NMNAT1−/− P53−/− RPE-1 if compared to the respective parental cells (FIG. 2c). These results are consistent with the observation that NMNAT1 loss had no impact on the proliferation of NMNAT1−/− P53+/+ cells To evaluate whether the catalytic activity of NMNAT1, i.e. nuclear NAD+, is essential for the survival of HRD cells, the inventors complemented NMNAT1−/− RPE-1 cells with either wild type (WT) or a catalytically-dead version of the enzyme (W169A, MUT) and tested cell survival upon BRCA2 knockdown. While complementation with WT NMNAT1 increased PARP1 activation, and thus was able to rescue the amount of DNA lesions in NMNAT1−/− cells to the same extent than in parental cells, the W169A mutant did not (FIG. 3a,b). In accordance with these results, both PARPi sensitivity and synthetic lethality with BRCA2 were rescued by WT NMNAT1 complementation, but not by W169A NMNAT1 (FIG. 3c,d). Altogether, these datas demonstrate that NMNAT1, a nuclear enzyme other than PARP1, is crucial for the survival of HRD cells. These results indicate that NMNAT1 is a key factor which activities are necessary for the survival of HRD cells.

Inhibition of NMNAT1 Kills PARP-Inhibitor and Cisplatin-Resistant BRCA1/2-Mutated Tumors, Including Those with Somatic Reversion of the BRCA1/2 Mutations

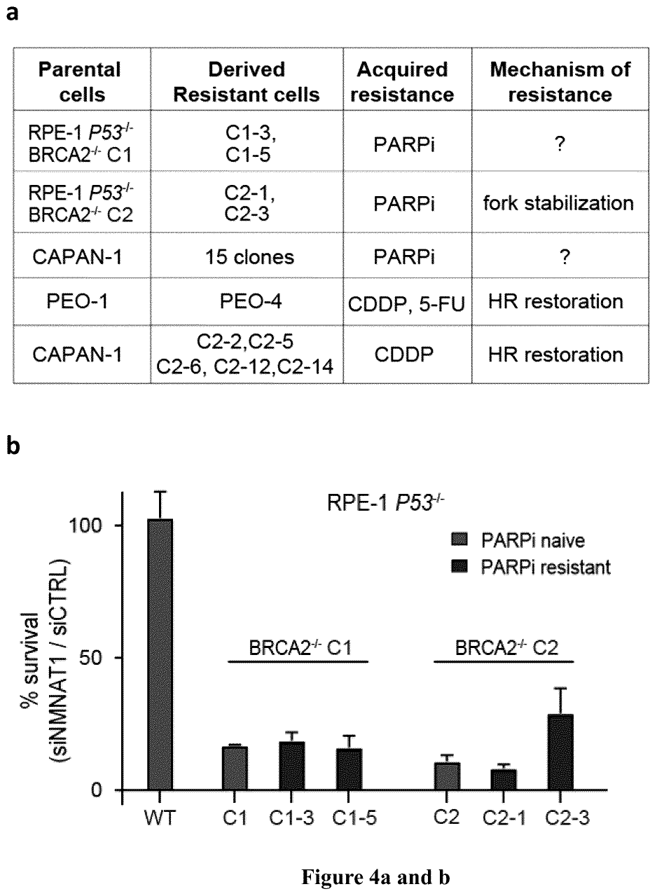

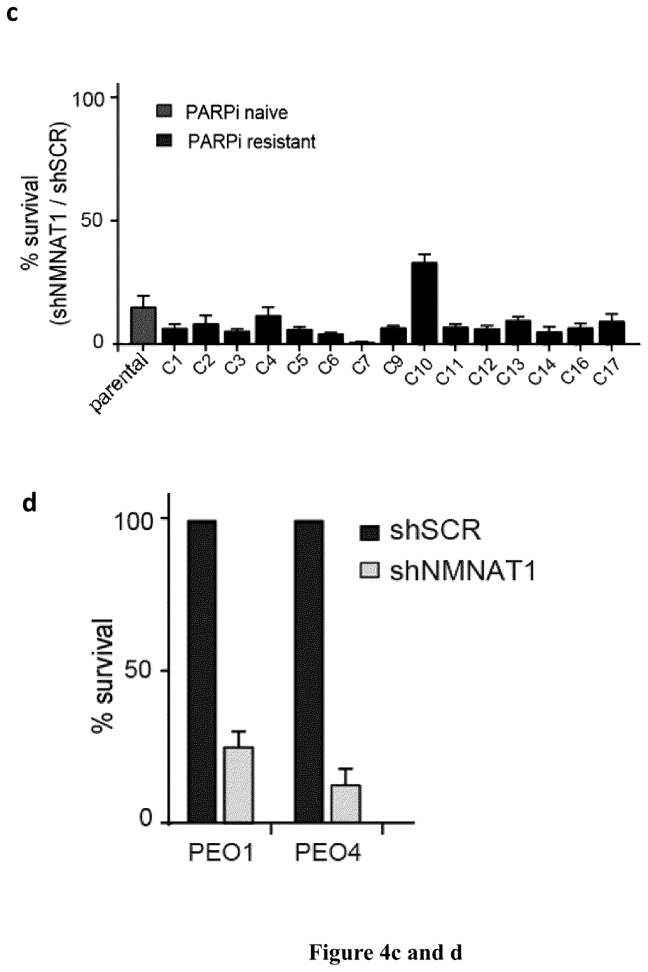

The inventors generated resistant cells by continuous exposure of BRCA2−/− RPE-1 to rucaparib. The inventors did not observe HR restoration in any of the derived clones, but resistance arose through fork stabilization. In those clones, NMNAT1 knockdown by siRNA impaired the clonogenic ability to a similar extent than that of the drug-naive BRCA2−/− cells, while sparing the HRP RPE-1 (FIG. 4b).

Then using a similar approach, several clones were derived from the BRCA2-mutated pancreatic cancer cell line CAPAN-1. After becoming resistant to rucaparib, those clones did not restore HR but rather developed resistance through other mechanisms that need to be further investigated. Knockdown of NMNAT1 impaired the clonogenic ability of all the CAPAN-1-derived resistant clones (FIG. 4c). Together, these data show that targeting NMNAT1 also kills PARPi-resistant HRD cells.

Nonetheless, HR restoration by secondary mutations in the BRCA genes is the only mechanism of resistance to PARPi validated so far in clinic. For this reason, the inventors evaluated the effect of NMNAT1 inhibition in HRD cells that developed chemo-resistance through BRCA2 secondary mutations restoring the open reading frame of the gene and thus HR. In particular, the inventors tested the chemo-resistant HR-restored ovarian cancer cell line PEO4—together with its BRCA2-mutated paired parental PEO1 cells—and five clones derived from prolonged in vitro cisplatin exposure of CAPAN-1 cells, each bearing different secondary mutation in BRCA2 gene. Surprisingly, shRNA-mediated knockdown of NMNAT1 impaired the colony formation ability of both PEO4 cells (FIG. 4d) and resistant CAPAN-1 clones, suggesting that targeting NMNAT1 kills chemo-resistant cells regardless the mechanism of drug resistance.

### Example 2: SIRT6 Inhibition Kills BRCA1 and BRCA2-Mutated Tumor Cells

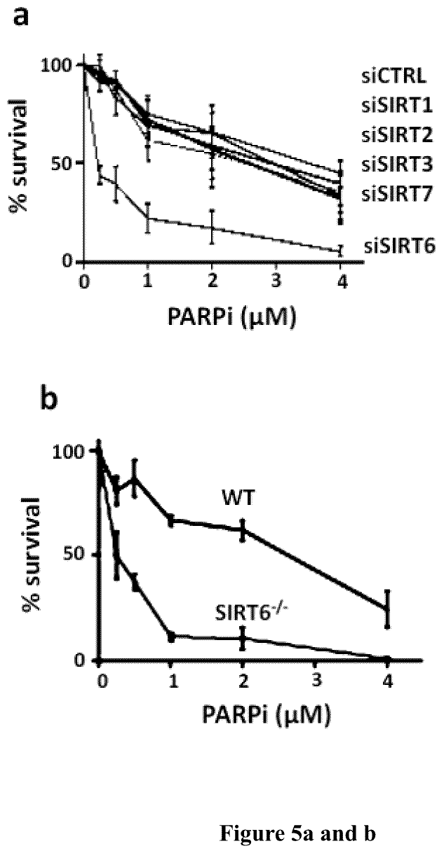

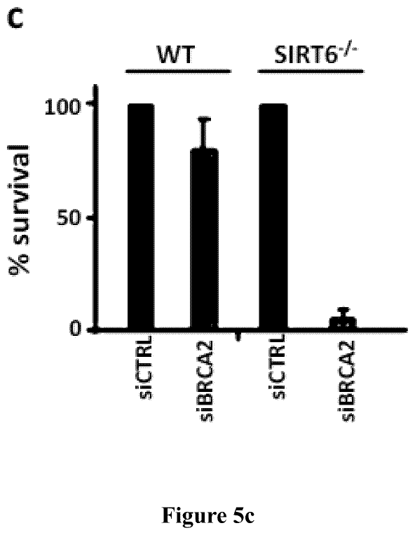

Besides PARP1, nuclear NAD+ is also used by sirtuins, a class of enzymes involved in many processes including DNA repair, to deacetylate and mono-ADP-ribosylate their substrates. To assess the role of the nuclear sirtuins (SIRT1, SIRT2, SIRT3, SIRT6 and SIRT7) the nuclear sirtuins were silenced by specific siRNA and tested PARPi response. Deletion of SIRT6, but not SIRT1, SIRT2, SIRT3 or SIRT7, sensitized cells to PARPi (FIG. 5a).

To corroborate these findings, the inventors generated SIRT6−/− RPE-1 cells and evaluated their survival in response to PARPi or BRCA2 knockdown. While having only a mild effect on RPE-1 cells, both PARPi and BRCA2 knockdown impaired the clonogenic ability of SIRT6−/− cells (FIG. 5b,c). To determine which catalytic activity of SIRT6 is important for the survival of HRD cells, the inventors complemented SIRT6−/− cells with either wild type or dissociation-of-function SIRT6 mutants, which were previously described (data not shown). While S56Y SIRT6, which lacks both activities, did not rescue either PARPi sensitivity or BRCA2 synthetic lethality, the MAR-dead mutant G60A and the deacetylase-dead mutant R65A did partially rescue, although to a lesser extent than the wild type SIRT6 (data not shown). Together, these data indicate that both catalytic activities of SIRT6 are essential for the survival of HRD cells through a PARP-independent mechanism.

Inhibition of SIRT6 Kills PARP-Inhibitor and Cisplatin-Resistant BRCA1/2-Mutated Tumors, Including Those with Somatic Reversion of the BRCA1/2 Mutations.

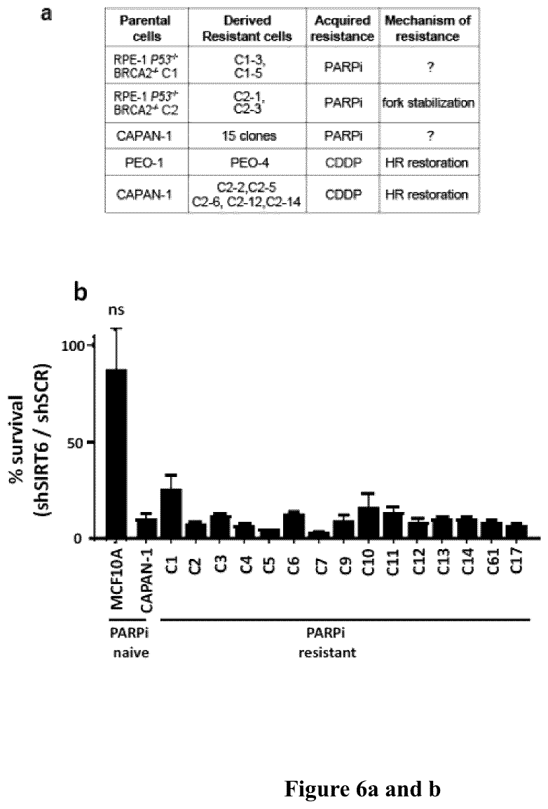

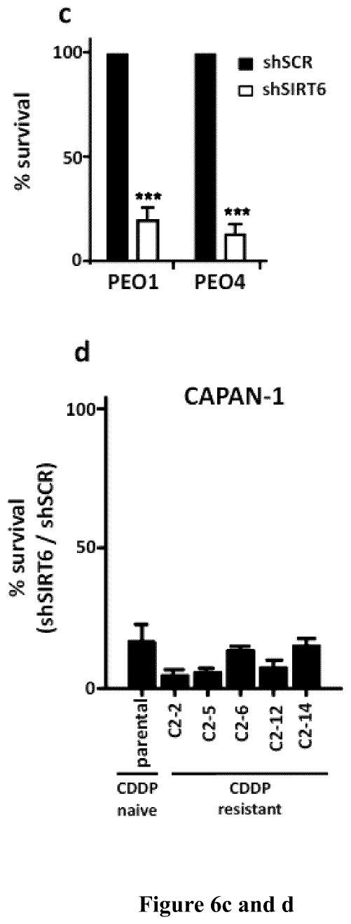

Despite the striking cytotoxic effect of PARPi in BRCA-mutated cells, insurgence of resistance is ubiquitous in clinic and calls for the design of alternative therapies for the treatment of advanced diseases. Our findings that inhibition of SIRT6 kills HRD cells in a PARP1-independent manner suggest that targeting this axis might also tackle BRCA-mutated cells that developed resistance to PARPi. To test this, the inventors used several cellular models, which recapitulated the major known mechanisms of resistance, including fork stabilization and HR restoration (FIG. 6a).

The inventors generated resistant cells by continuous exposure of BRCA-associated cancer cells to rucaparib. Several clones were derived from the BRCA2-mutated pancreatic cancer cell line CAPAN-1. After becoming resistant to rucaparib, those clones did not restore HR but rather developed resistance through other mechanisms. Knockdown of SIRT6 impaired the clonogenic ability of all the CAPAN-1-derived resistant clones (FIG. 6b). Together, these data show that targeting SIRT6 also kills PARPi-resistant HRD cells.

Nonetheless, HR restoration by secondary mutations in the BRCA genes is the only mechanism of resistance to PARPi validated so far in clinic. For this reason, the inventors evaluated the effect of SIRT6 inhibition in HRD cells that developed chemo-resistance through BRCA2 secondary mutations restoring the open reading frame of the gene and thus HR. In particular, the inventors tested the chemo-resistant HR-restored ovarian cancer cell line PEO4—together with its BRCA2-mutated paired parental PEO1 cells—and five clones derived from prolonged in vitro cisplatin exposure of CAPAN-1 cells, each bearing different secondary mutation in BRCA2 gene. Surprisingly, shRNA-mediated knockdown of SIRT6 impaired the colony formation ability of both PEO4 cells (FIG. 6c) and resistant CAPAN-1 clones (FIG. 6d), suggesting that targeting SIRT6 kills chemo-resistant cells regardless the mechanism of drug resistance.

### The SIRT6 Inhibitor OSS_128167 Kills PARPi Naïve and Resistant Tumor Cells.

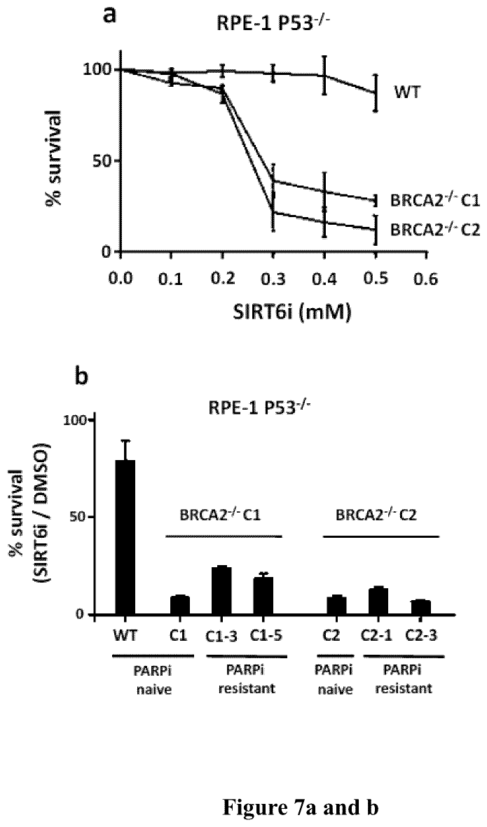

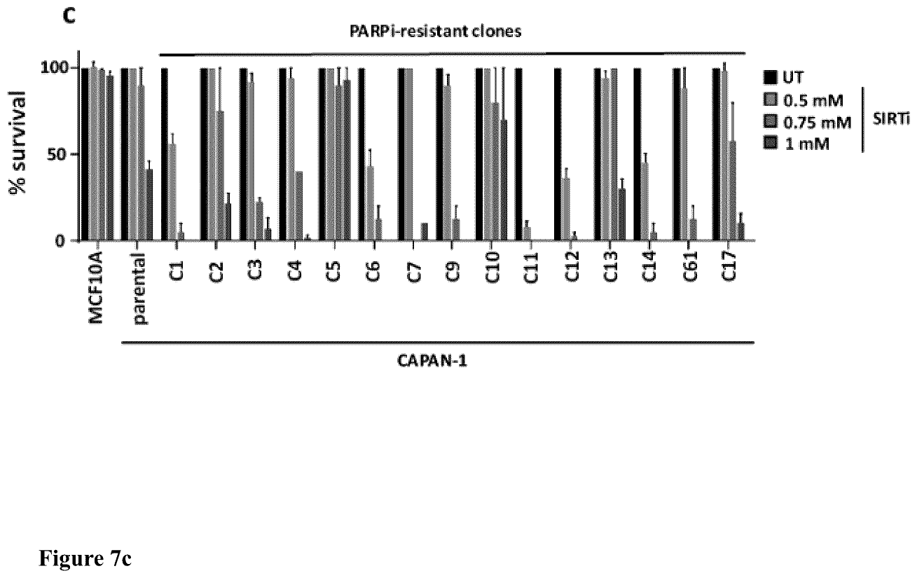

Inhibition of SIRT6 by OSS_128167 impaired the clonogenic ability of BRCA2−/− RPE-1 cells, while having no effect on the survival of the parental HRP cells (FIG. 7a), thus confirming the synthetic lethality between SIRT6 and BRCA. Furthermore, the SIRT6 inhibitor also killed the PARPi-resistant BRCA2−/− RPE-1 cells to a similar extent than the parental drug-naïve cells (FIG. 7b). Likewise, SIRT6 inhibition resulted in cell death also in the PARPi-resistant clones that were generated by continuous drug exposure of the BRCA2-mutated pancreatic cancer cell line CAPAN-1 (FIG. 7c). Importantly, at the same doses OSS_128167 did not affect the clonogenic ability of the normal breast epithelial cell MCF10A, indicating that SIRT6 inhibition is not harmful for normal cells. Altogether, these data suggest that targeting SIRT6 catalytic activity by OSS_128167 might be a valuable strategy to tackle PARP inhibitor naïve and resistant HRD tumor cells.

### The SIRT6 Inhibitor Kills Chemo-Resistant BRCA-Associated Cancer Cells in an In Vivo Mouse Model of Patient-Derived Xenograft (PDX) BRCA1-Mutated Triple Negative Breast Cancer (TNBC) Resistant to PARPi.

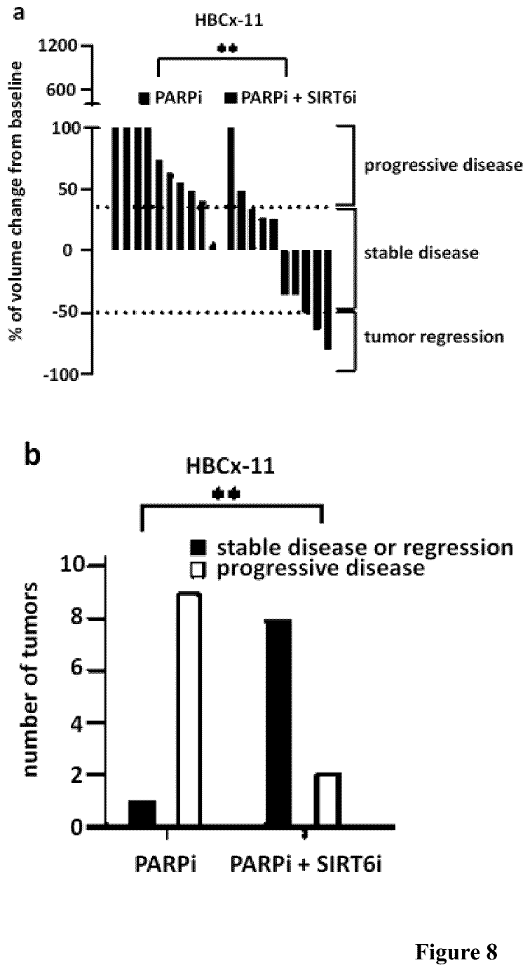

The inventors used the patient-derived xenograft (PDX) HBCx-11 model, established from a BRCA1-mutated triple negative breast cancer (TNBC) resistant to PARPi. The HBCx-11 tumor was transplanted in nude mice, which were further separated in groups receiving either vehicle, PARPi or the combination of PARPi+SIRT6i. While PARPi alone had almost no effect on tumor regression (1 tumor out of 10 showed a response), the inventors found that the addition of the SIRT6i to the chemotherapy dramatically enhanced the tumor response (8 tumor out of 10) (FIG. 8).

### Example 3: UNG Functions Downstream of SIRT6 in the Survival of BRCA1/2-Mutated in Parental (PARPi-Naive) and PARPi-Resistant Tumors

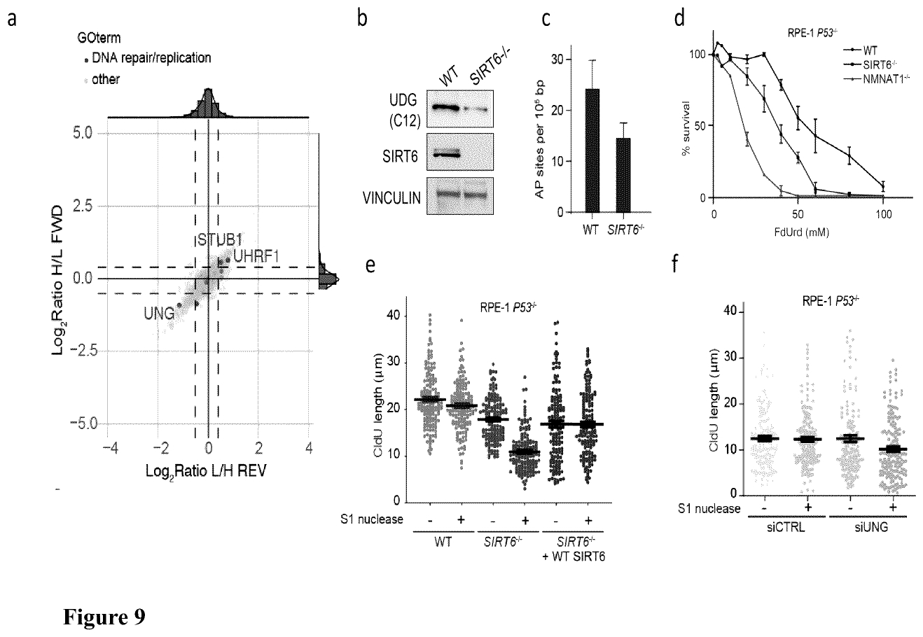

To discover the mechanism by which SIRT6 sustains HRD cell survival, the inventors undertook a comprehensive SILAC (Stable isotope labeling by amino acids in cell culture)-based proteomic approach to identify novel SIRT6 targets. The comparison of WT and SIRT6−/− nuclear proteomes led to the identification of proteins, which expression was either down- or up-regulated in SIRT6−/− cells (FIG. 9a, b). SIRT6 could sustain genome stability through the regulation of key DNA repair/replication proteins, which function would be essential for HRD cell survival. To test this hypothesis, the inventors performed Gene Ontology (GO) analysis of our proteomic dataset and observed that the DNA glycosylase UNG was down-regulated in SIRT6−/− cells.

The inventors found that that SIRT6−/− cells show more abasic sites in their genome, which is compatible with a reduce UNG activity in these cells (FIG. 9c). Surprisingly, they found that depletion of either NMNAT1 or SIRT6 exquisitely sensitizes cells to FdUrd, a thymidylate synthase inhibitor, which trigger uracil misincorporation in the genome (FIG. 9d). These data suggest a defect in uracil excision (i.e. UNG activity) in the absence of NMNAT1 and/or SIRT6. Finally, they found that depleting UNG by siRNA affects replication fork stability as shown by the increased number of ssDNA gaps (FIG. 9f). To note, ssDNA gaps were also increased in SIRT6−/− cells further suggesting that UNG is a target of SIRT6 (FIG. 9e).

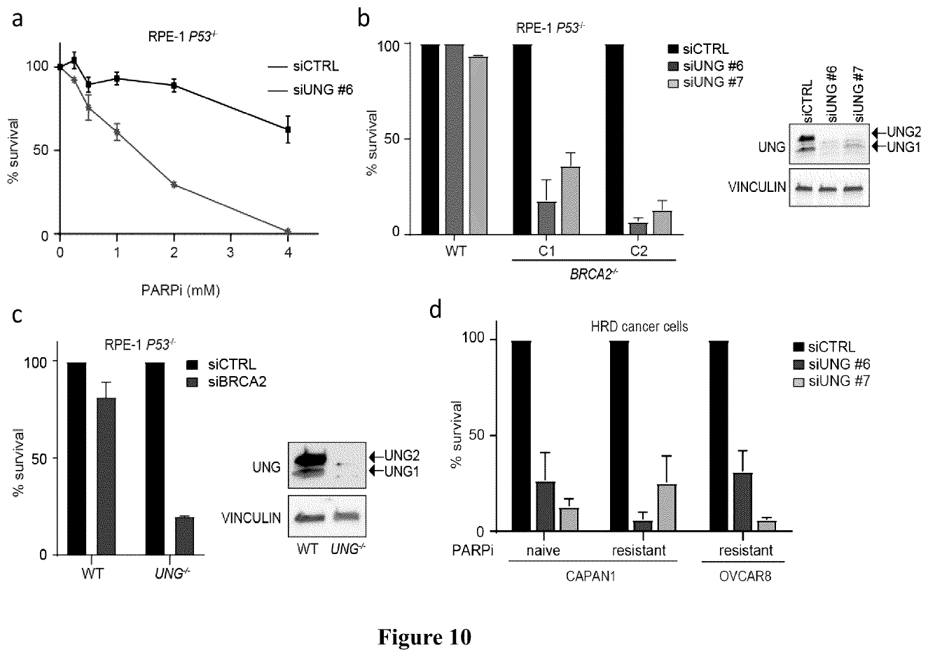

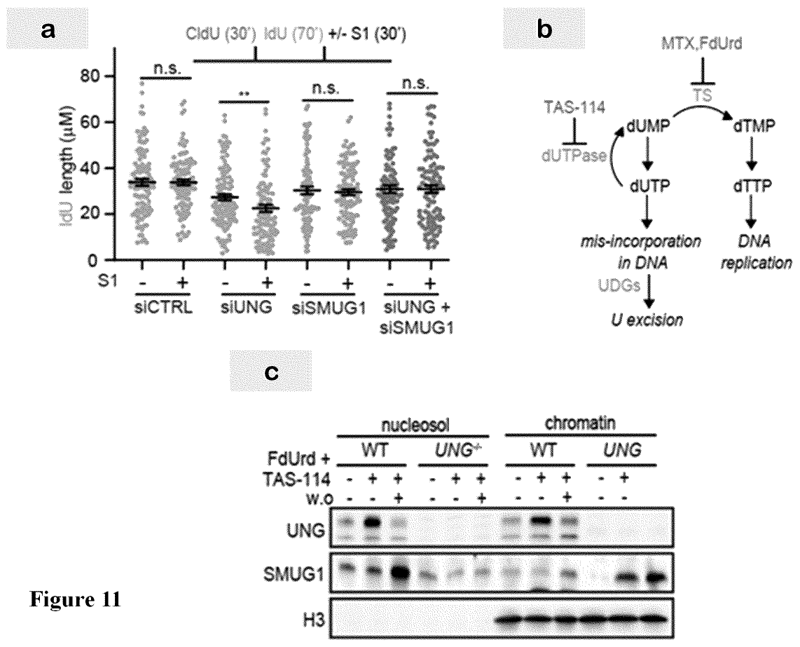

Since the inventors found that UNG activity is deficient in the absence of SIRT6, they next wonder whether UNG inhibition phenocopied NMNAT1/SIRT6 inhibition in the response to PARPi and HRD cell survival. They found that, RPE-1 P53^{-/-} cells knockout or depleted for UNG were sensitized to rucaparib in comparison with parental cells (FIG. 10a). Next, they tested the role of UNG in HRD cell fitness, they knocked-down UNG by two different siRNAs in both WT and BRCA2^{-/-} RPE-1 clones and tested cell survival in clonogenic assays. The results show that UNG silencing kills BRCA2^{-/-} clones, while sparing HR-proficient (HRP) cells (FIG. 10b). Similarly, UNG^{-/-} cells were sensitive to BRCA2 depletion (FIG. 10c). Based on the data, they tested whether targeting UNG would also kill HRD cancer cells with acquired resistance to PARPi. To that purpose, they generated rucaparib-resistant ovarian cancer cells (OVCAR8: clone RR14) and evaluated their survival following UNG knockdown. They found that UNG siRNA killed both parental OVCAR8 and the clone RR14 with acquired PARPi resistance. In addition, they found that UNG inhibition killed PARPi-resistant clones derived from BRCA2^{-/-} cells, as well as those derived from the BRCA2-mutated CAPAN1 tumor cell lines (FIG. 10d).

Overall, the data unveiled a key role of UNG in HRD cell survival and cell response to PARPi, which phenocopies SIRT6/NMNAT1 functions. The functional results indicate that targeting UNG could represent a valuable therapeutic approach for HRD tumors, including those with acquired PARPi resistance.

### Example 4: UNG-Mediated Uracil Excision is Regulated by Nuclear NAD⁺

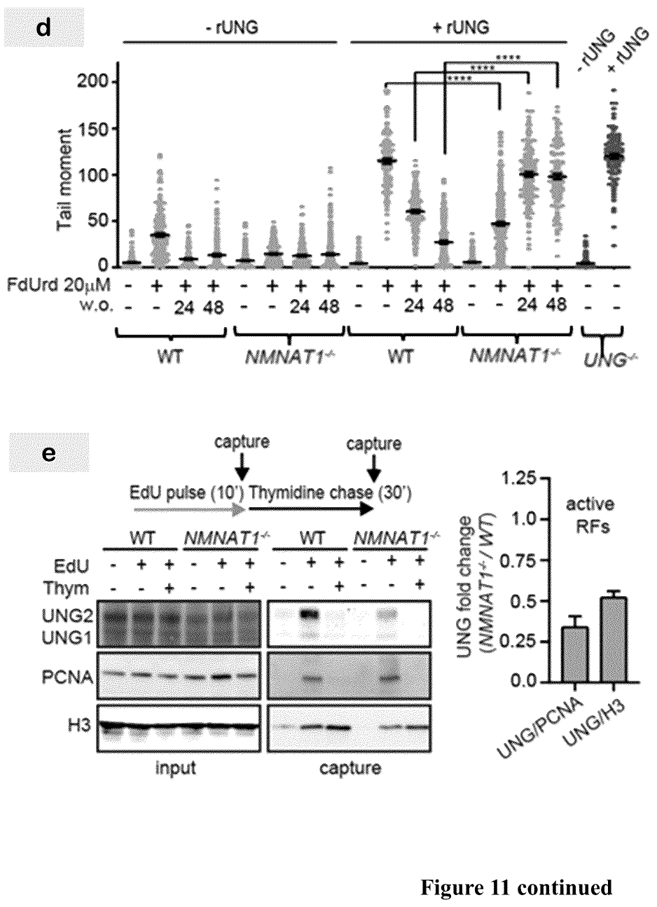

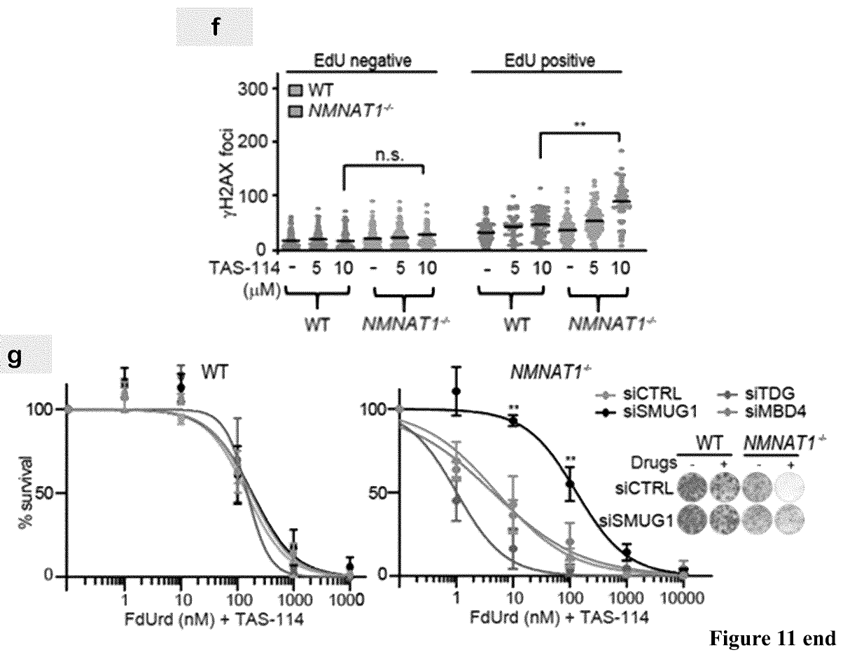

The uracil DNA glycosylase SMUG1 was recently shown to create single-stranded DNA (ssDNA) gaps following translation synthesis inhibition. Hence, the inventors tested whether the basal processing of gDNA-uracil by either UNG or SMUG1 might also result in ssDNA gap formation. They quantified ssDNA gaps with a modified DNA fiber assay, based on the use of the ssDNA specific endonuclease S1. The inventors found that siRNA-mediated depletion of UNG slows down the replication fork (RF) speed and results in post-replicative ssDNA gaps, whereas depletion of SMUG1 does not. Nonetheless, SMUG1 knockdown rescued the ssDNA gaps observed upon UNG loss, indicating that SMUG1 generates gaps in the absence of UNG (FIG. 11a). Typically, ssDNA gaps arise from replication repriming behind an obstacle that interferes with RF progression. The inventors hypothesized that the observed SMUG1-mediated gap formation could be due to SMUG1 binding to gDNA-uracil accumulation in the absence of UNG, which leads to cycles of RF stalling and repriming.

To investigate this hypothesis, the inventors generated UNG knockout (UNG^{-/-}) in RPE-1 TP53^{-/-} cells and examined the distribution of the two UDGs in the soluble (nucleosol) and insoluble (chromatin) fractions. To force dU misincorporation in gDNA without inducing thymidine starvation, they combined low doses of the thymidylate synthase (TS) inhibitor 5-fluorodeoxyuridine (FdUrd) with the dUTPase inhibitor TAS-114 (FIG. 11b). The low dose of FdUrd (0.1 μM) we used only partially inhibited TS and thus moderately decreased the dTTP pool, but—in combination with TAS-114 that increased the dUTP level at the same time—it resulted in gDNA-uracil accumulation without cell cycle arrest (FIG. 15a left panel and 15b). Upon gDNA-uracil increase, the inventors observed that the absence of UNG recruitment to chromatin in UNG^{-/-} cells was accompanied by a sustained retention of SMUG1 on chromatin, whereas in WT cells SMUG1 accumulated in the soluble nuclear fraction (FIG. 11c).

### UNG and SMUG1 Interplay in Uracil Excision Under the Control of Nuclear NAD⁺

To investigate the mechanism underlying the interplay between UNG and SMUG1, the inventors investigated the role of nuclear NAD⁺ (nicotinamide adenine dinucleotide), a molecule with essential—yet poorly characterized—functions in DNA repair. In the nucleus of mammalian cells NAD⁺ is synthesized by NMNAT1 (nicotinamide mononucleotide adenylyltransferase 1) and fuels the activity of PARP1 and sirtuins (FIG. 15c). The inventors generated NMNAT1 knockout (NMNAT1^{-/-}) RPE-1 cells and evaluated their sensitivity to a panel of DNA damaging agents (Table 1). NMNAT-1^{-/-} cells were more sensitive than WT cells to drugs targeting the TS pathway (ie. FdUrd and MTX) and further sensitized to TS inhibitors when combined with TAS-114 (FIG. 15d-f). The observation that NMNAT1^{-/-} cells were more than a 100-fold more sensitive than WT cells to FdUrd+TAS-114 co-treatment points towards a specific role of nuclear NAD⁺ in the regulation of gDNA-uracil homeostasis rather than being part of a general response to replicative stress (FIG. 15f). Further strengthening this, the inventors found that—similarly to UNG^{-/-} cells—NMNAT1^{-/-} cells exhibited increased gDNA-uracil and decreased survival upon targeting of DUT, either by siRNA-mediated knockdown (siDUT) or pharmacological inhibition (TAS-114) (FIG. 15 g-i).

TABLE 1
Calculated IC₅₀ of WT and NMNAT1^{-/-} cells
for the indicated DNA-damage inducing drugs

| | IC₅₀ | IC₅₀ | Fold change |
|---|---|---|---|
| Drug | WT | NMNAT1^{-/-} | (WT/NMNAT1^{-/-}) |
| MTX (μM) | 112.2 | 8.8 | 12.75 |
| PARPi (μM) | 2.57 | 0.86 | 2.99 |
| FDURD (μM) | 51.55 | 17.65 | 2.92 |
| HU (mM) | 1.566 | 0.6367 | 2.46 |
| MMS (mM) | 0.2419 | 0.1298 | 1.86 |
| IR (gy) | 3.6 | 2.781 | 1.29 |
| H202 (μM) | 22.84 | 22.12 | 1.03 |

To further explore the role of nuclear NAD⁺ in the control of uracil excision, the inventors quantified gDNA-uracil in WT and NMNAT1^{-/-} cells by using a modified alkaline COMET assay in which permeabilized nuclei are incubated with recombinant UNG (rUNG) and the resulting DNA breaks are used as a readout of gDNA-uracil. The inventors observed an increased uracil content following treatment with high dose of FdUrd (20 μM), which induced cell cycle arrest in both cell lines because of thymidine starvation (FIG. 16a). This increase was more pronounced in WT than NMNAT1^{-/-} cells, likely due to the reduced proliferation (and consequent lower rate of dU misincorporation) of NMNAT1^{-/-} cells. Nevertheless, after drug washout, uracil content returned to basal levels in wild-type (WT) cells whereas it further increased in the NMNAT1^{-/-} cells (FIG. 11d).

Accumulation of uracil in NMNAT1^{-/-} cells can be a consequence of either persisting dU misincorporation, and/or of defective uracil excision. Mass spectrometry-based metabolic analysis revealed a slight increase of free dTTP and a decrease of dUMP in NMNAT1^{-/-} as compared to WT cells, ruling out a higher rate of dU misincorporation as the cause of gDNA-uracil increase (FIG. 16b).

The UNG2 nuclear isoform travels along the active replication forks (RFs) together with PCNA and RPA and cleaves newly incorporated uracil during DNA replication. Hence, to test the possibility that nuclear NAD⁺ regulates the presence of UNG2 specifically at RFs, the inventors performed an immunoblot analysis of UNG after isolation of proteins on nascent DNA (iPOND). The inventors found a significant decrease of UNG2 level at active RFs in NMNAT1^{-/-} cells when compared to WT cells, whereas no changes in PCNA level were observed (FIG. 11e). Additionally, FdUrd treatment triggered the recruitment of the nuclear isoform UNG2 to chromatin, which was impaired in NMNAT1^{-/-} cells (FIG. 15j, top panel). This was not due to differences in either TS inhibition or cell cycle distribution (FIG. 15j).

Collectively, these data reveal a crucial interplay between the two major UDGs that is required for the maintenance of genomic stability. UNG is crucial for efficient DNA replication, and in its absence, the resulting accumulation of gDNA-uracil engages SMUG1 on chromatin. Nonetheless, the attempted compensation operated by SMUG1 leads to decreased speed fork, accumulation of ssDNA post-replicative gaps and genomic instability. Finally, the inventors found that this interplay is regulated by nuclear NAD⁺, which sustains the presence of UNG at RFs and restrains SMUG1 in non-replicating chromatin to ensure efficient uracil excision.

### SMUG1 Creates DSBs During Replication in the Absence of UNG-Mediated Uracil Excision.

The inventors next investigated the consequences of gDNA-uracil accumulation on genomic stability. In cells lacking either NMNAT1 or UNG, the increase of gDNA-uracil by TAS-114 triggered DNA double-strand break (DSB) formation exclusively during DNA replication (as shown by increased number of γH2AX foci in EdU-positive cells) (FIGS. 11f and 16c, d). These data are in sharp contrast with previous works showing that the presence of uracil itself in the DNA has no detrimental consequences on RF progression and genomic stability. Hence, the inventors hypothesized that the recognition and/or excision of uracil by other UDGs, rather than the presence of uracil, results in toxic DNA intermediates that impair RF progression in cells with reduced UNG.

Similarly to what is observed in UNG^{-/-} cells, the inventors found that the impairment of UNG recruitment to chromatin in NMNAT1^{-/-} cells upon gDNA-uracil accumulation was accompanied by a sustained retention of SMUG1 on chromatin (FIG. 16e). Notably, the inventors found that knockdown of SMUG1 completely rescues the sensitivity of NMNAT1^{-/-} cells to gDNA-uracil increasing drugs, while knockdown of the other human UDGs does not (FIG. 11g).

Altogether, these data show that when UNG-mediated excision of uracil is lost or reduced, chromatin trapping of SMUG1 interferes with RF progression, resulting in ssDNA gap and DSB formation thus conferring heightened sensitivity to gDNA-uracil inducing drugs.

### Example 5: UNG Inhibition Leads to SMUG1-Mediated DNA Damage and HR Activation

### Overcoming UNG Excision Capability Results in SMUG1-HMCES Trapping on Chromatin, Sustained PARP1 Hyperactivation and HR Activation

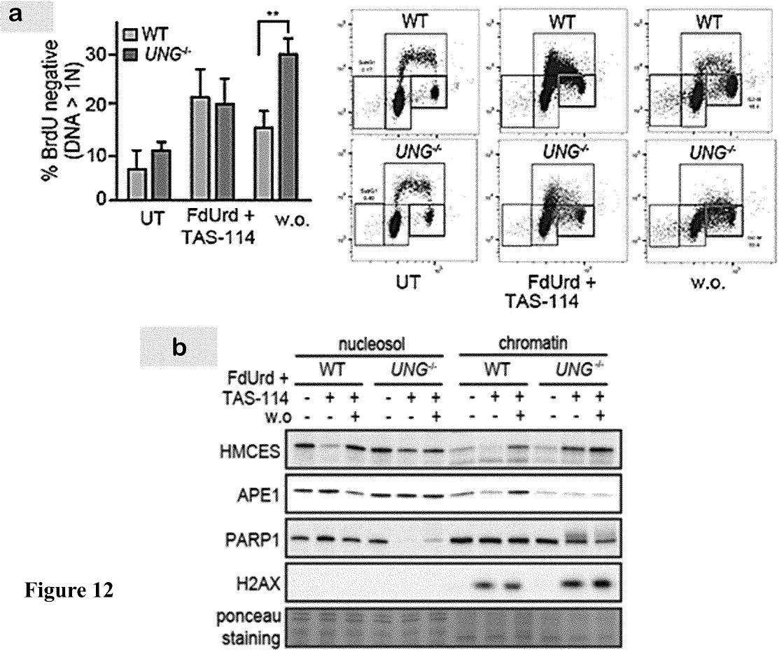

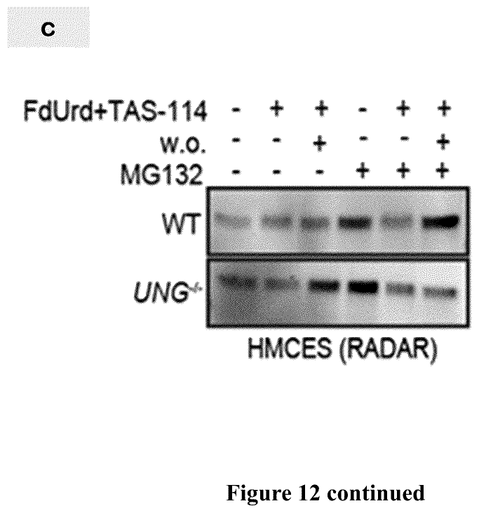

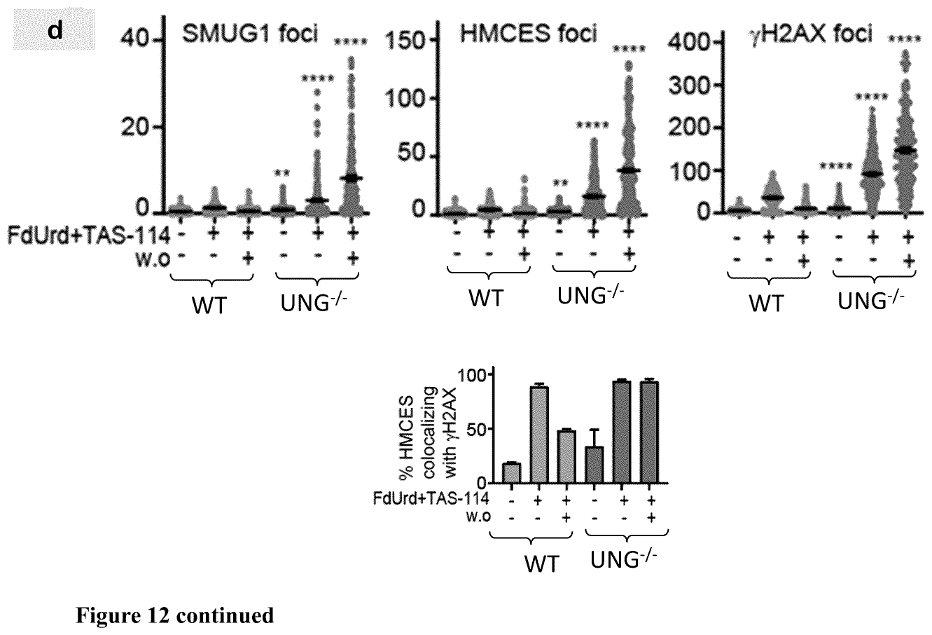

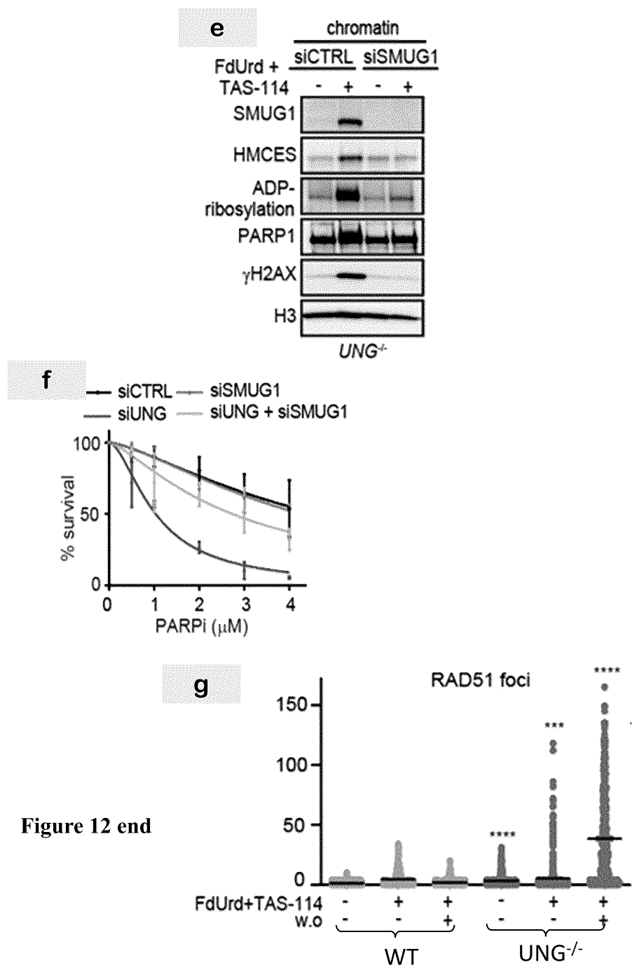

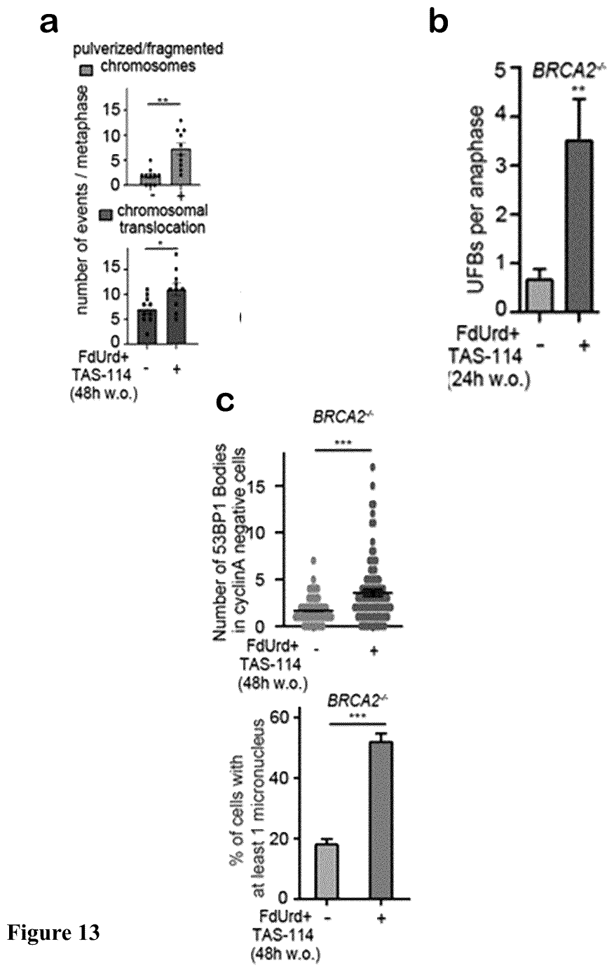

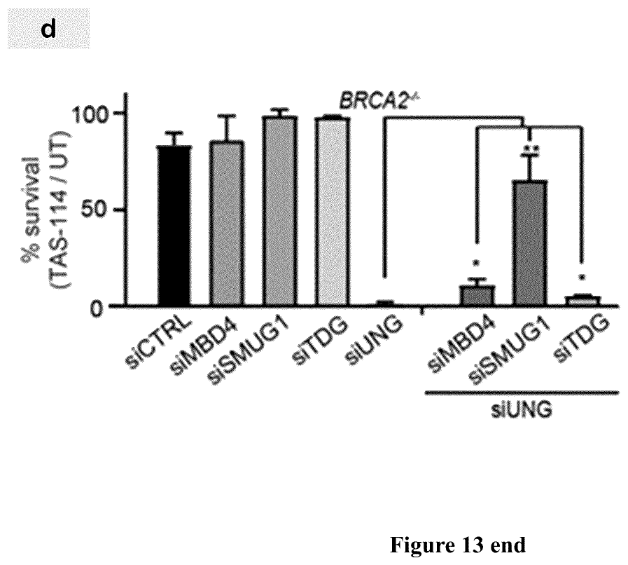

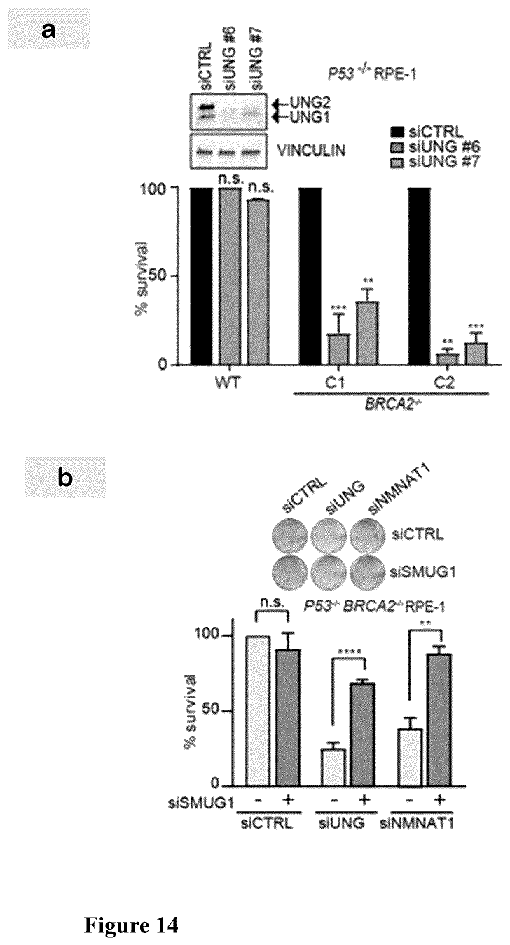

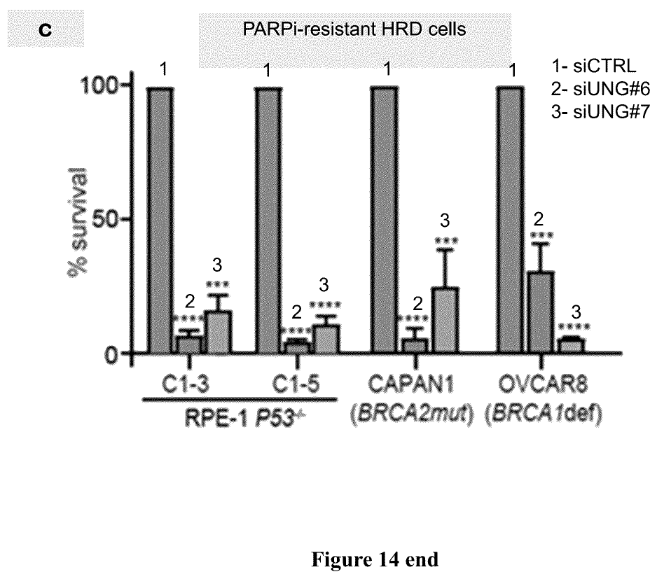

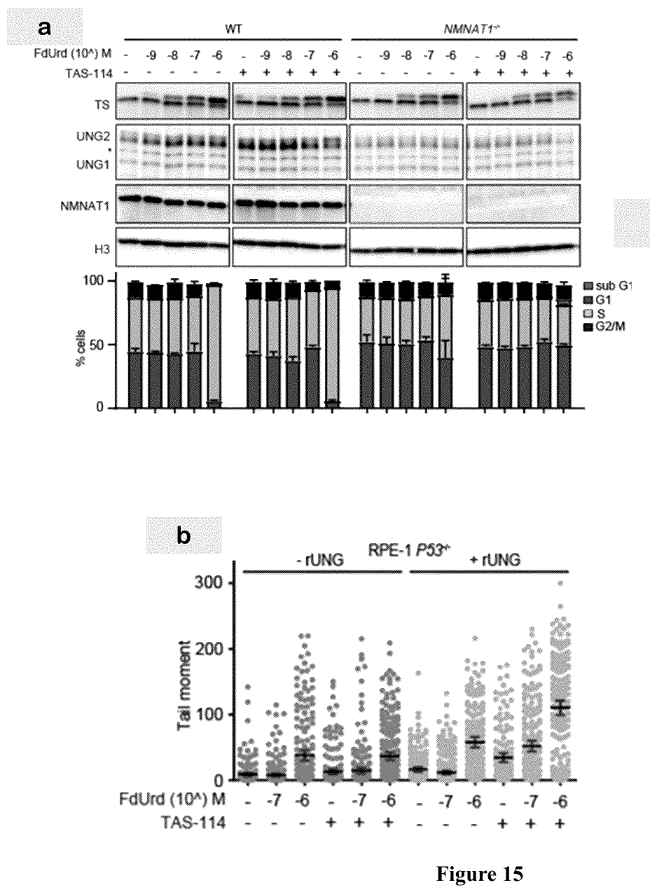

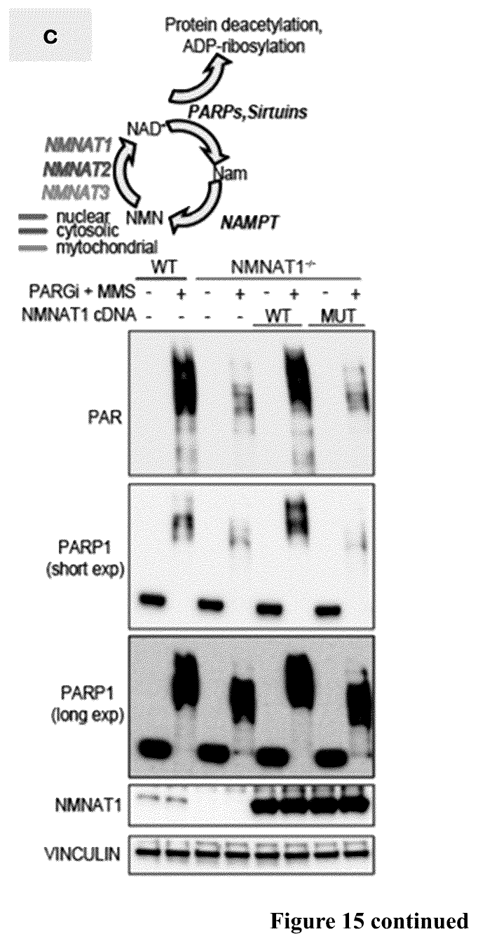

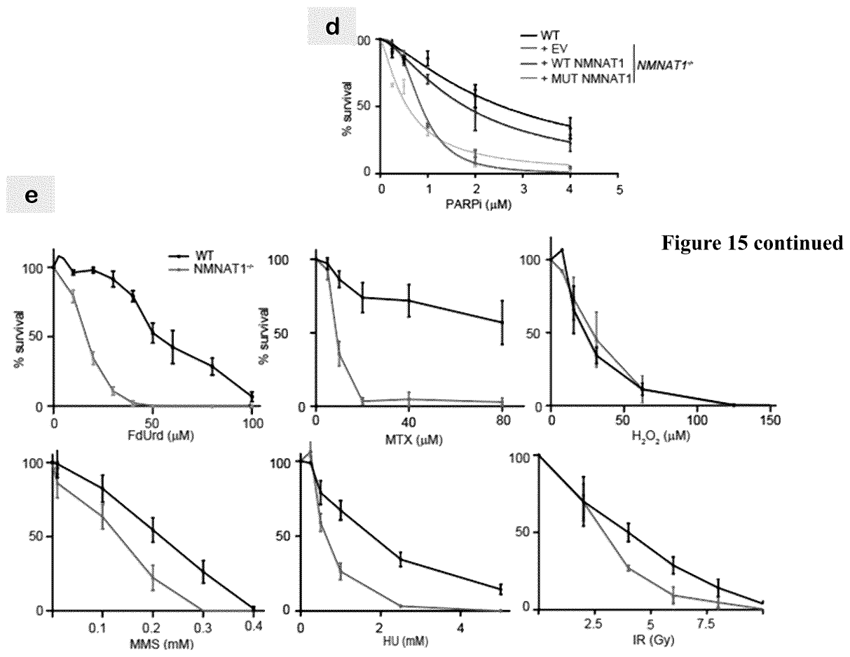

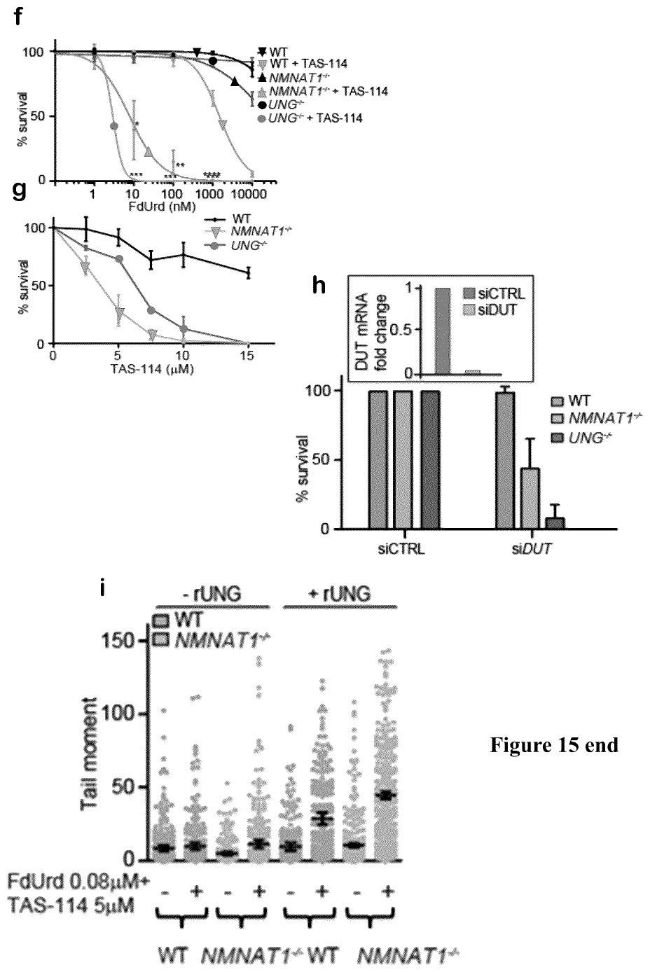

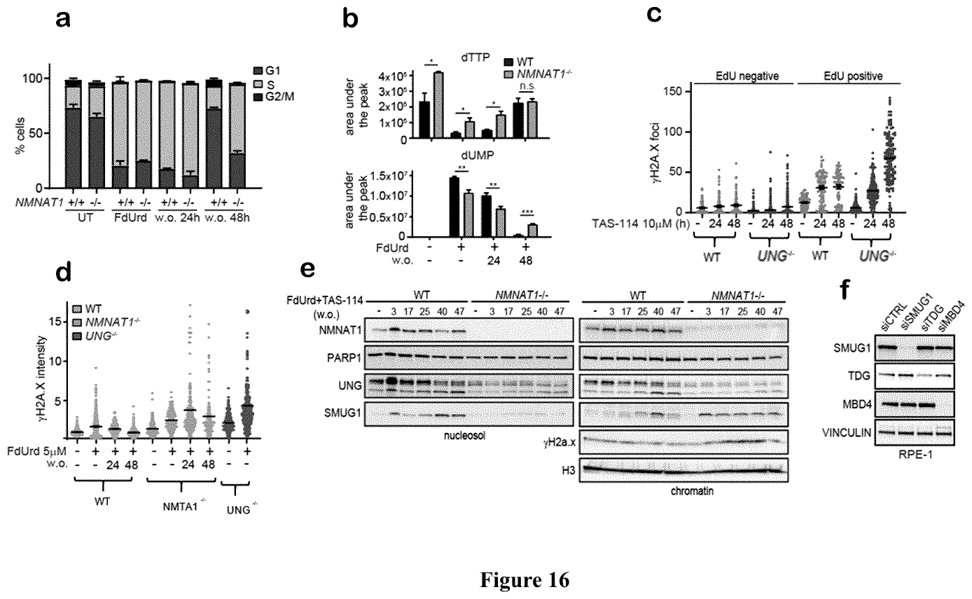

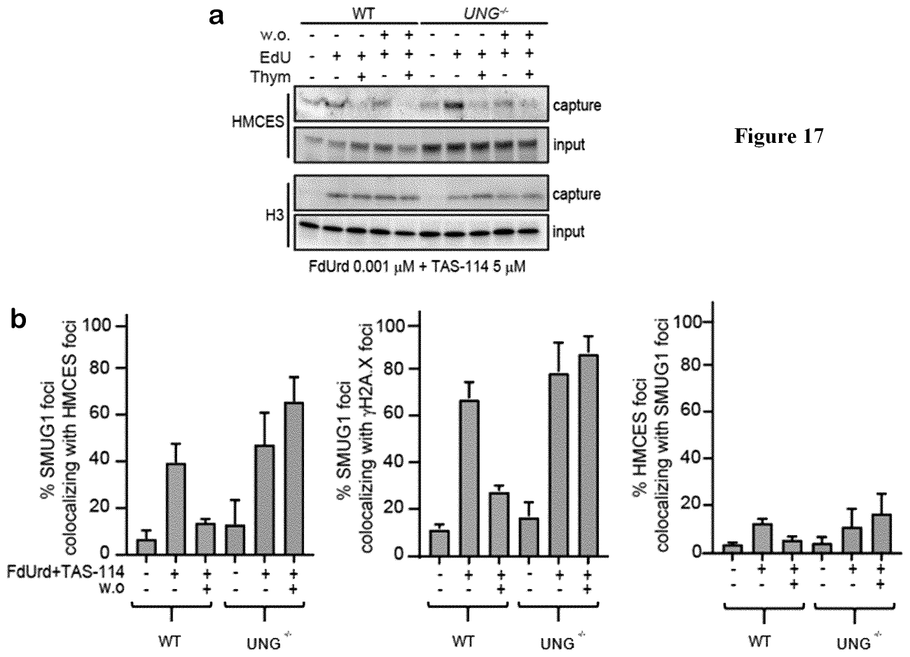

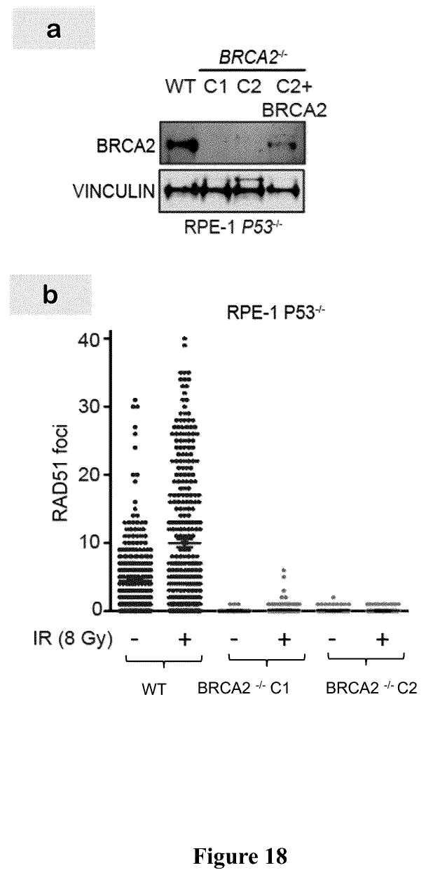

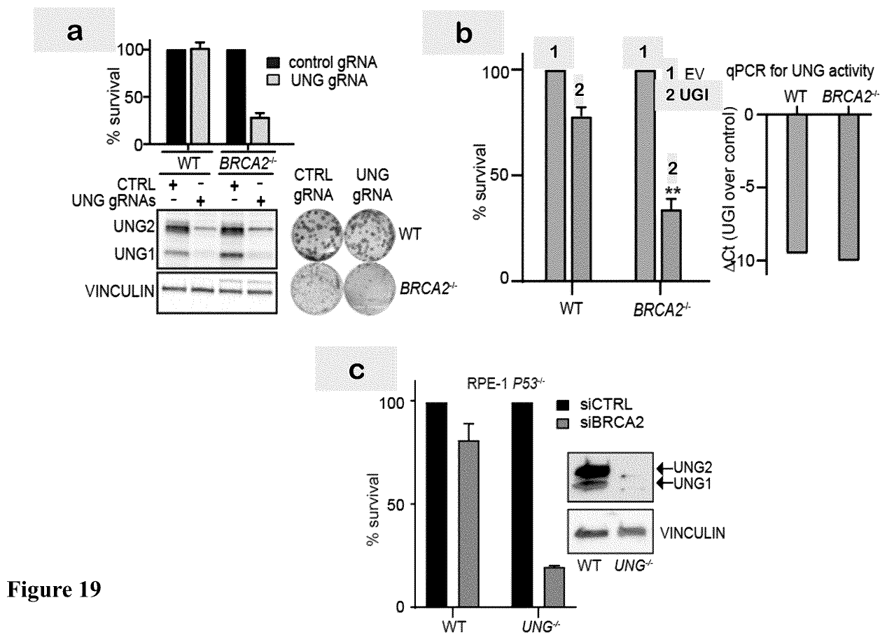

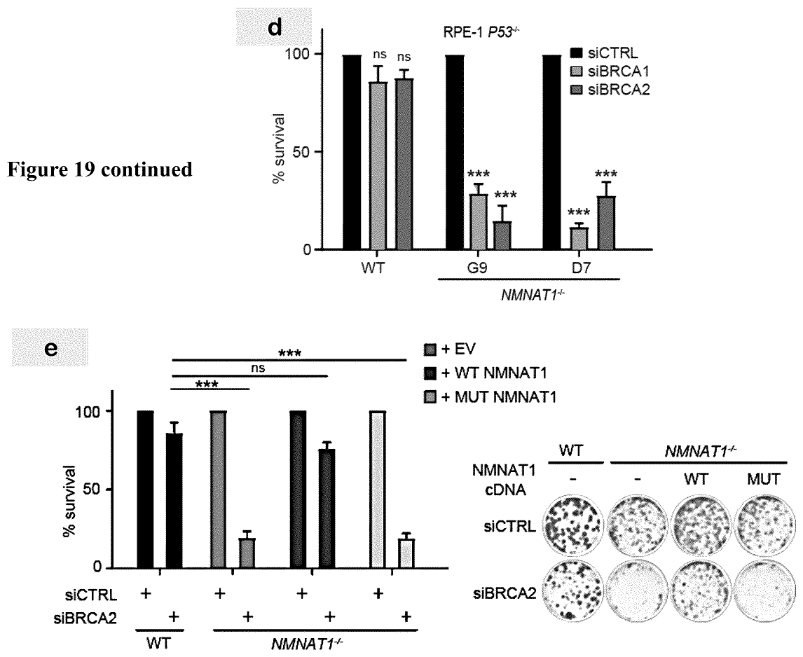

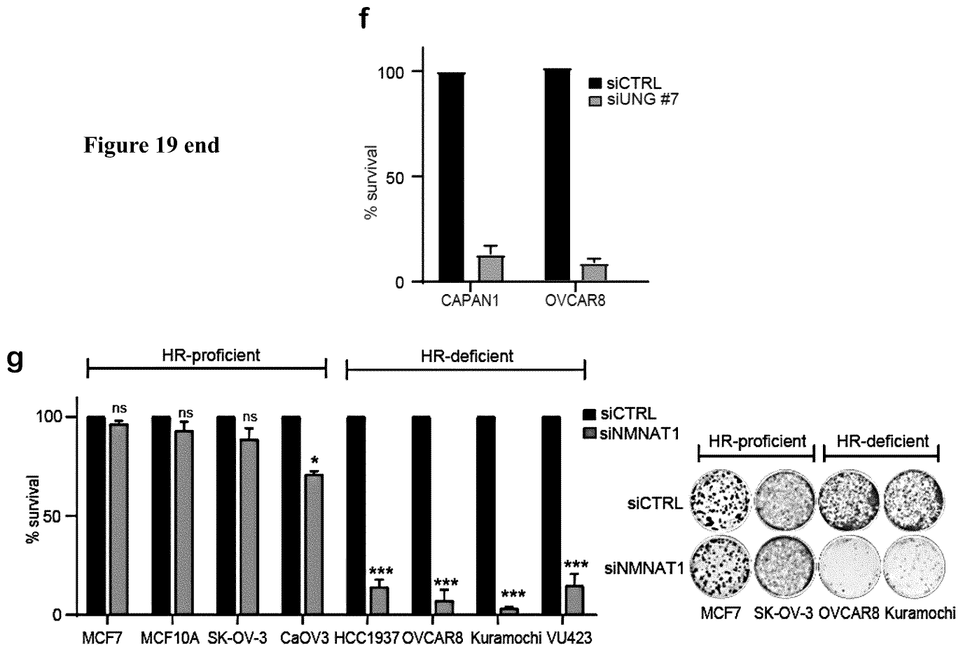

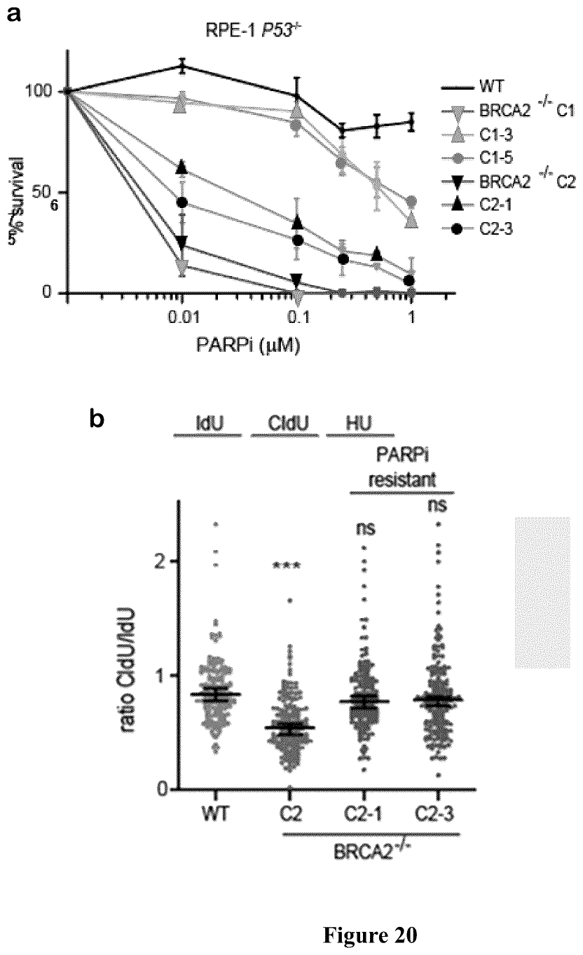

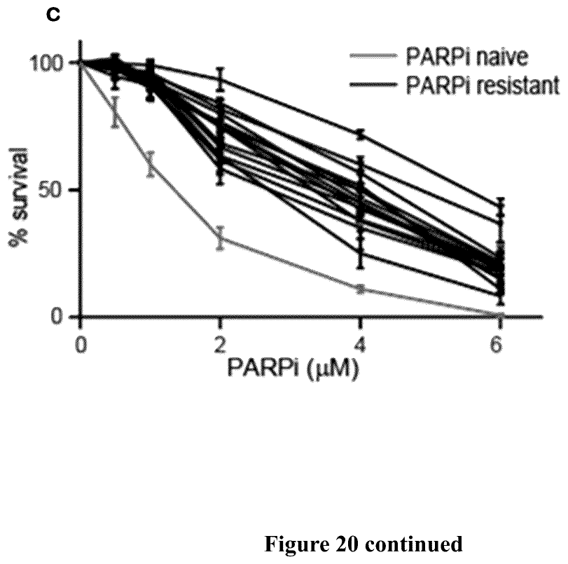

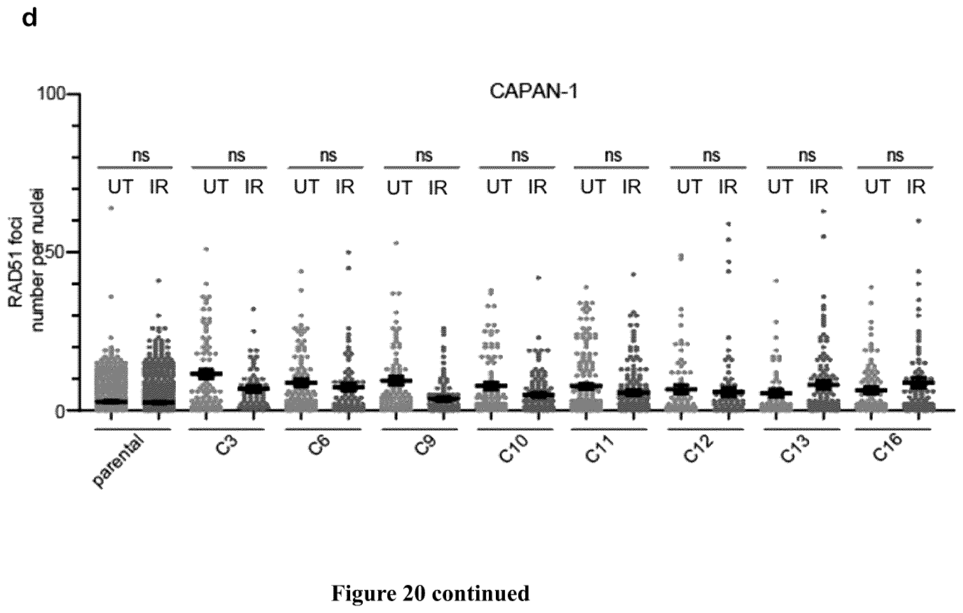

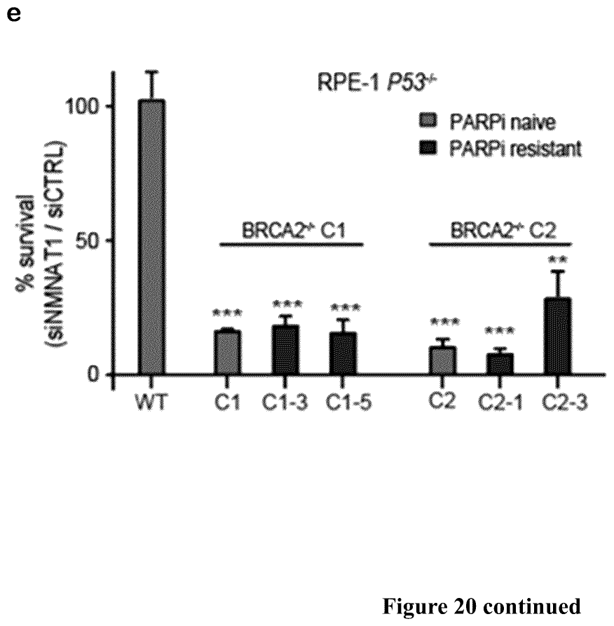

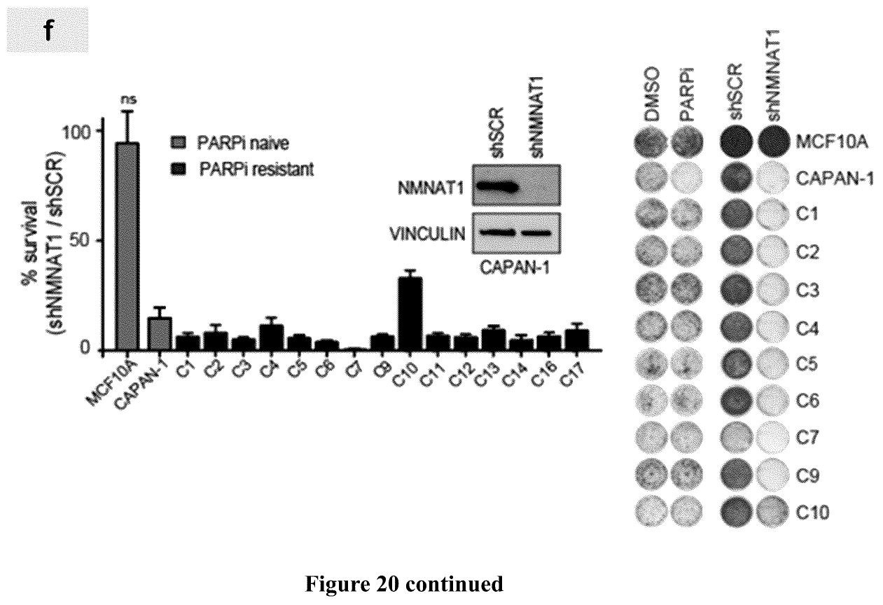

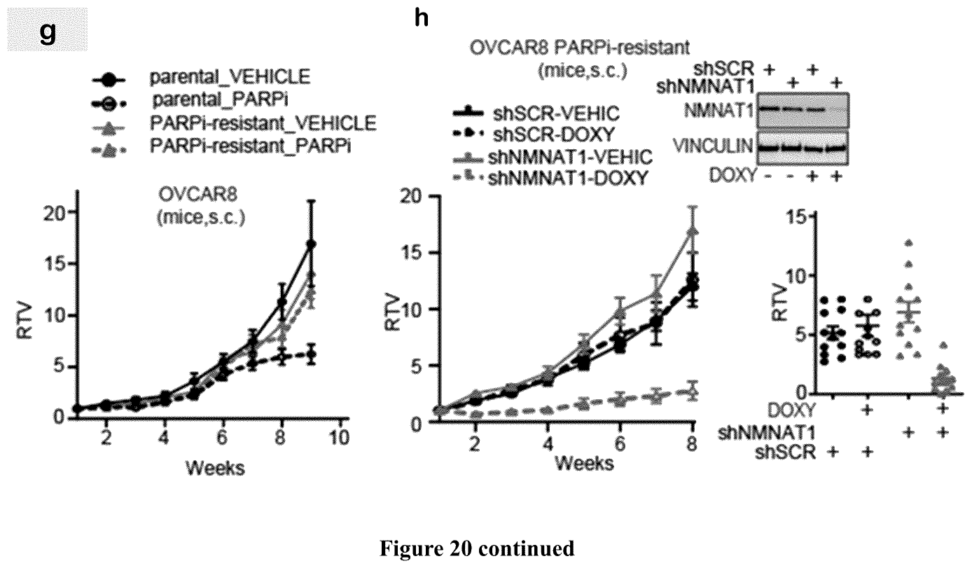

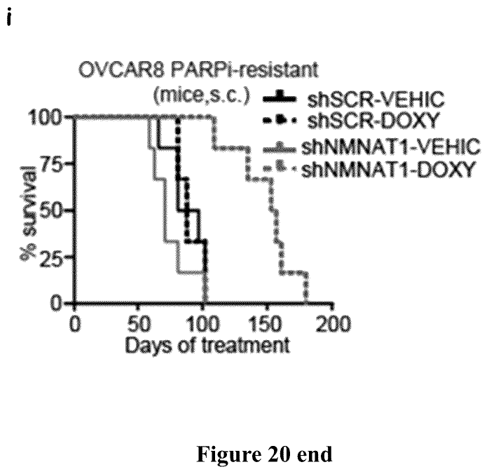

To investigate the mechanism by which SMUG1 exerts its toxicity in the absence of UNG, the inventors challenged UNG^{-/-} cells with gDNA-uracil increasing drugs (FdUrd+TAS-114). Cell cycle analysis revealed that gDNA-uracil accumulation slows down DNA replication in both WT and UNG^{-/-} RPE-1 cell lines. However, UNG^{-/-} cells were not able to resume DNA replication after drug washout as opposed to WT cells, as quantified by the percentage of BrdU-negative cells (FIG. 12a).

SMUG1 has a stronger avidity than UNG for apurinic/apyrimidinic (AP) sites generated upon uracil excision. Thus, SMUG1 persistent binding to AP sites might compromise their processing through base-excision repair (BER) and represent a roadblock for RF progression. Of note, the inventors found that trapping of SMUG1 on chromatin was associated with that of the 5-hydroxymethylcytosine (5hmC) binding, ES cell-specific (HMCES), a recently described sensor of AP sites (FIG. 12b).

HMCES covalently binds to AP sites during replication at ssDNA-dsDNA junctions (i.e. RFs) forming stable DNA-protein crosslinks (DPCs), which shield the AP sites from aberrant processing to maintain genome integrity. However, HMCES levels at RFs were only slightly increased in UNG^{-/-} cells, suggesting that the observed retention of HMCES mainly occurs in non-replicating chromatin (FIG. 17a). To evaluate whether HMCES forms DPCs in UNG^{-/-} cells, the inventors quantified DNA-protein adducts by using the rapid approach to DNA adduct recovery (RADAR) assay. Notably, UNG^{-/-} cells showed higher levels of HMCES-DNA adducts both at basal level and after drug exposure (FIG. 12c).

Next, the inventors investigated whether the increase of SMUG1 and HMCES occurs in the same genomic regions (i.e., AP sites). To this date, HMCES and SMUG1 have never been reported to form foci. Surprisingly, the inventors detected massive formation of SMUG1 and HMCES colocalizing foci in UNG^{-/-} cells only following dU misincorporation, suggesting that HMCES crosslinks to AP-sites created by SMUG1 (FIGS. 12d and 17b). To validate this, the inventors knocked down SMUG1 in UNG^{-/-} cells and assessed chromatin-bound HMCES levels. Depletion of SMUG1 completely abolished HMCES recruitment to chromatin, confirming that HMCES associates with SMUG1-generated AP sites (FIG. 12e).

For the correct processing of uracil by BER, AP sites must be incised by APE1, the AP site-specific endonuclease. The inventors found that retention of SMUG1 and HMCES on chromatin is associated on the one hand to impaired recruitment of APE1, and on the other hand to prolonged PARP1 activation (identified by ADP-ribosylation) and increased DSB formation (identified by γH2AX) (FIG. 12b). The inventors next reasoned that prolonged activation and chromatin retention of PARP1 resulting from SMUG1 trapping might sensitize cells to PARPi. In accordance with this observation, the inventors found that depletion of UNG sensitizes HR-proficient (HRP) cells to the PARPi rucaparib and that concomitant depletion of SMUG1 rescues drug sensitization (FIG. 12f).

The impaired recruitment of APE1 suggests that BER is unable to access and process the SMUG1/HMCES-bound DNA lesions. Of note, we found a basal and gDNA-uracil-mediated increase in RAD51 foci (a readout of HR activity) in UNG^{-/-} as compared to WT cells (FIG. 12g left). Importantly, most of the SMUG1 foci colocalized with RAD51 and γH2AX (FIG. 12g right) and depletion of SMUG1 abolished ADP ribosylation and γH2AX induction in UNG^{-/-} cells (FIG. 12e). This suggests that the HR pathway is activated to rescue the DNA lesions caused by SMUG1 trapping. These results show that in the absence of efficient uracil excision by UNG at RFs, SMUG1-generated AP sites are shielded by HMCES and their processing by APE1 through BER is impaired. Therefore, persistent SMUG1/HMCES-DNA adducts create a roadblock for RF progression, resulting in DNA lesions that trigger HR-mediated repair. In this view, the trapping of SMUG1 does not compensate for the loss of UNG but rather leads to genome instability and death in cells with high gDNA-uracil.

### dU Misincorporation Results in Under-Replicated DNA and Chromosomal Fragmentation in HR-Deficient Cells.

Next, the inventors examined the consequences of SMUG1-HMCES trapping at misincorporated dU on the fitness of BRCA1/2-deficient (i.e. HRD) cells. The inventors used BRCA2^{-/-} clones (C1 and C2) previously generated in RPE-1 TP53^{-/-} cells (FIG. 18a, b).

To enhance the replication stress induced by dU, the inventors combined TAS-114 to low doses of FdUrd. First, they measured chromosomal breaks in BRCA2^{-/-} cells by inducing premature chromatin condensation by calyculin A upon dU misincorporation. Multicolor FISH revealed that FdUrd+TAS-114 treatment of BRCA2^{-/-} cells induces a strong increase in chromosomal rearrangements, including breaks and fragmentation (FIG. 13a). Chromosomal fragmentation upon premature DNA condensation likely arises from under-replicated DNA (UR-DNA) stretches. Mitotic entry in the presence of UR-DNA has been shown to initiate mitotic DNA synthesis (MiDAS). UR-DNA that escapes MiDAS resolution forms DNA bridges in anaphase known as ultra-fine bridges (UFBs) and may be inherited by the daughter G1 cells by encapsulation in 53BP1 nuclear bodies (53BP1 NBs) and micronuclei. The inventors observed that FdUrd+TAS-114 co-treatment of BRCA2^{-/-} cells induces a 3 to 4-fold increase in the number of UFBs as well as 53BP1 NBs and micronuclei as quantified in anaphase and cyclin A-negative G1 phase, respectively (FIG. 13b, c). In addition, UNG depletion sensitized BRCA2^{-/-} cells to low dose of TAS-114, which was also rescued by knockdown of SMUG1, and none of the other UDGs, further reinforcing our data showing that SMUG1 trapping creates DNA lesions that activate HR-mediated repair (FIG. 13d).

Altogether, these data imply that the fine-tuned regulation of UNG/SMUG1 interplay in uracil excision is crucial for the maintenance of genomic stability in BRCA2-deficient cells.

### Example 6: Targeting Uracil Excision by UNG Kills HR-Deficient Tumors, Regardless of their Resistance to PARPi

### Synthetic Lethality Between UNG Inhibition and BRCA1/2 Mutations

Next, the inventors evaluated a potential synthetic lethality between UNG depletion and BRCA1/2 mutations. The inventors found that UNG (or NMNAT1) inhibition by either siRNAs or CRISPR sgRNA or the peptide inhibitor UGI selectively kills BRCA2^{-/-} cells (clones C1 and C2) (FIGS. 14a and 19a, b). Likewise, BRCA1/2 depletion by siRNA selectively impaired the clonogenic survival of NMNAT1^{-/-} and UNG^{-/-} cells (FIG. 19c, e). Notably, the inventors observed that loss of UNG (or NMNAT1) killed HR-deficient cancer cell lines while sparing HRP cancer cells, further confirming a synthetically lethal interaction between UNG and BRCA1/2, and NMNAT1 and BRCA1/2 (FIG. 19f, g). Finally, depletion of SMUG1 rescued the synthetic lethality between BRCA2and NMNAT1/UNG (FIG. 14B).

Altogether, these data show that accumulation of gDNA-uracil—by either increased dU misincorporation or inhibition of UNG-mediated excision—leads to SMUG1-dependent UR-DNA, chromosomal fragmentation, and ultimately HRD cancer cell death.

### Targeting UNG-Mediated Uracil Excision Kills a Subset of PARPi-Resistant HR-Deficient Tumors

Despite the initial success of PARPis in the treatment of BRCA-deficient tumors, the development of resistance has become a clinical challenge. The inventors explored the potential of targeting uracil excision as a novel strategy to overcome PARPi resistance. The inventors generated PARPi-resistant clones by continuous exposure of two different BRCA2^{-/-} RPE-1 clones to rucaparib. First, they confirmed that these generated clones developed drug resistance, which we found to be associated with increased RF stability (FIG. 20a, b).

To evaluate whether disrupting UNG-mediated uracil excision represents a valuable strategy to kill PARPi-resistant HRD cells, the inventors generated several PARPi-resistant clones from either the BRCA1-deficient (promoter hypermethylation) ovarian cell line OVCAR8 and the pancreatic BRCA2-mutated cell line CAPAN1. None of the CAPAN1 clones acquired PARPi resistance through HR restoration, as quantified by RAD51 foci formation (FIG. 20 c, d). Clonogenic survival assays showed that knockdown of either UNG or NMNAT1 specifically decreased the survival of all PARPi-resistant HRD cancer cells tested (FIGS. 14c and 20e, f). Next, to apply these findings into a translational setting, the inventors employed organoids derived from patient-derived xenografts (PDXs), established from triple negative breast cancers (TNBCs). Finally, the inventors evaluated the impact of UNG inhibition—by knocking down NMNAT1—on the PARPi-resistant HRD tumor growth in vivo. PARPi-resistant BRCA1-deficient tumor cells (OVCAR8, clone 14) expressing either doxycycline-inducible NMNAT1 or scrambled (Scr) shRNA were xenotransplantated into athymic nude mice. Tumor growth was monitored upon PARPi treatment and/or NMNAT1 depletion. PARPi slowed down the growth of the parental OVCAR8 tumors but had no impact on the growth of the PARPi-resistant clone (FIG. 20 f). Nonetheless, NMNAT1 depletion significantly impaired PARPi-resistant OVCAR8 tumor growth and increased survival of mice bearing tumors (FIG. 20h, i).

Collectively, these data show that a subset of PARPi-resistant HRD tumors, including some with acquired RF stability or HR restoration, remain sensitive to the accumulation of gDNA-uracil. Thus, the inventors demonstrated that UNG-mediated uracil excision constitutes a novel vulnerability of HRD tumors with potential for overcoming PARPi resistance.

What is claimed is:

1. A method of treating resistant Homologous Recombination Deficiency (HRD) cancer in a subject in need thereof, comprising administering to the subject a therapeutically effective amount of a Uracil DNA glycosylase (UNG) inhibitor, wherein the UNG inhibitor is a molecule capable of silencing the gene expressing the UNG enzyme or a molecule inhibiting UNG enzymatic properties.

2. The method according to claim 1, wherein the resistant HRD cancer is resistant BRCA-associated cancer, chemo-resistant HRD cancer, chemo-resistant BRCA-associated cancer or metastatic resistant HRD cancer.

3. The method according to claim 1, wherein the resistant HRD cancer is PARPi resistant BRCA-associated cancer or cisplatin resistant BRCA-associated cancer, including those with somatic reversion of the BRCA mutation and HR restoration.

4. The method according to claim 1, wherein said UNG inhibitor is a small organic molecule, a polypeptide, an aptamer, an oligonucleotide or an antibody.

5. The method according to claim 4, wherein said oligonucleotide is an antisense oligonucleotide, a siRNA, a shRNA, a DNA aptamer or a RNA aptamer.

6. The method according to claim 1, in combination with a PARP inhibitor.

7. The method according to claim 6, wherein the PARP inhibitor is selected from the group consisting of olaparib, rucaparib, niraparib, talazoparib, iniparib, veliparib, Pamiparib (BGB-290), CEP 9722, E7016, E7449 and 3-Aminobenzamide.

8. The method according to claim 1, in combination with cisplatin.

9. A method of treating resistant Homologous Recombination Deficiency (HRD) cancer in a subject in need thereof, comprising administering to the subject a pharmaceutical composition comprising a Uracil DNA glycosylase (UNG) inhibitor and a pharmaceutical acceptable carrier, wherein the UNG inhibitor is a molecule capable of silencing the gene expressing the UNG enzyme or a molecule inhibiting UNG enzymatic properties.

10. The pharmaceutical composition for use according to claim 9, wherein the resistant HRD cancer is resistant BRCA-associated cancer, chemo-resistant HRD cancer or chemo-resistant BRCA-associated cancer.

11. A pharmaceutical composition comprising a Uracil DNA glycosylase (UNG) inhibitor in combination with a PARP inhibitor and a pharmaceutical acceptable carrier.

12. (canceled)
